(19) United States
(12) Patent Application Publication
(10) Pub. No.: US 2012/0028970 A1
(43) Pub. Date: Feb. 2, 2012

(54) HETEROARYLTHIOMETHYL PYRIDINE DERIVATIVE

(75) Inventors: Minoru Kameda, Chiba (JP); Kensuke Kobayashi, Ibaraki (JP); Chisato Nakama, Tokyo (JP); Makoto Ando, Kanagawa (JP); Nagaaki Sato, Saitama (JP)

(73) Assignee: MSD K.K., Tokyo (JP)

(21) Appl. No.: 13/263,809

(22) PCT Filed: Apr. 27, 2010
(86) PCT No.: PCT/JP2010/057847

§ 371 Date: Oct. 10, 2011

(30) Foreign Application Priority Data
Apr. 28, 2009 (JP) 2009-109631

(51) Int. Cl. A61K 31/541; C07D 451/04; C07D 401/14; C07D 413/14; A61K 31/4545; A61K 31/46; A61K 31/5377; A61P 9/12; A61P 9/10; A61P 13/12; A61P 9/00; A61P 25/00; A61P 25/08; A61P 25/22; A61P 25/24; A61P 25/32; A61P 25/28; A61P 3/04; A61P 3/10; A61P 27/06; A61P 5/00; C07D 417/14

(52) U.S. Cl. 514/227.8; 544/60; 546/125; 546/193; 544/124; 514/318; 514/304; 514/236.8

(57) ABSTRACT

The present invention relates to a compound represented by a formula (I): wherein X is a group represented by or the like; Y is a group represented by or the like; and Ar₁ is a group represented by or a pharmaceutically acceptable salt thereof.

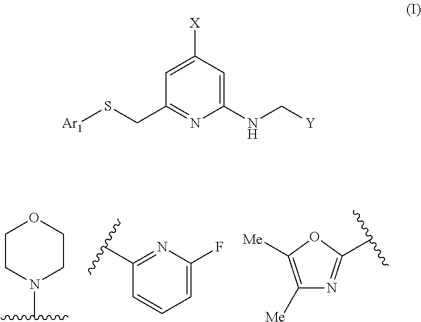

### FIELD OF THE INVENTION

The present invention relates to neuropeptide Y receptor antagonists which contain a heteroarylthiomethylpyridine derivative as an active ingredient. Furthermore, the present invention relates to a novel heteroarylthiomethylpyridine derivative.

### BACKGROUND OF THE INVENTION

Neuropeptide Y (hereinafter referred to as NPY), which is a peptide containing 36 amino acids, was first isolated in 1982 from porcine brain by Tatemoto et al. [for example, non-patent document 1]. NPY is widely distributed in the central and peripheral nervous systems and has a variety of in vivo actions as one of the peptides most abundantly present in the nervous system. That is, in the central nervous system, NPY acts as an aperitive and significantly promotes a fat accumulation via secretion of various hormones or actions of the nervous system. A continuous intracerebroventricular administration of NPY has been known to induce obesity and insulin resistance based on the above actions. NPY is also associated with emotional control and the actions of the central autonomic nervous system. In addition, in the peripheral nervous system, NPY is present together with norepinephrine in the sympathetic nerve ending and associated with the tonicity of the sympathetic nervous system. A peripheral administration of NPY has been known to cause vasoconstriction and enhance actions of other vasoconstrictors including norepinephrine (for example, non-patent document 2, non-patent document 3, non-patent document 4, non-patent document 5). The action of NPY is expressed when it is bound to an NPY receptor present in the central or peripheral nervous system. Therefore, inhibition of the binding of NPY to the NPY receptor allows prevention of the expression of the action of NPY. Consequently, compounds antagonizing the binding of NPY to the NPY receptor can be expected to be useful in the prevention or treatment of various diseases associated with NPY, for example, cardiovascular diseases such as hypertension, arteriosclerosis, nephropathy, cardiac diseases and angiospasm; diseases of central nervous system such as bulimia, depression, epilepsy, anxiety, alcoholism and dementia; metabolic diseases such as obesity, diabetes mellitus and hormone abnormality, or glaucoma (for example, non-patent document 6).

In addition, derivatives structurally similar to NPY are described to be bound to an NPY receptor and antagonize the activity of NPY (for example, patent document 1, patent document 2, non-patent document 7).

In addition, certain peptides have been recently found to inhibit binding of NPY to an NPY receptor (patent document 3, patent document 4).

However, such peptidic compounds have big problems in developing the compounds as medicaments. That is, such high-molecular-weight peptides generally have in vivo instability and are also short-acting.

Furthermore, such compounds are included in a compound group that can be hardly expected to have oral absorbability and intracerebral transferability.

In contrast, certain nonpeptidic compounds have been recently found to inhibit binding of NPY to an NPY receptor and antagonize the activity of NPY (for example, patent document 5, patent document 6).

However, such nonpeptidic NPY antagonists are structurally different from a compound according to the present invention and give no clue to the present invention.

In addition, as a compound having an antagonistic action to NPY Y1, for example, the following compound (A):

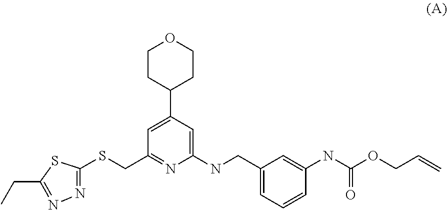

is disclosed (patent document 7).

However, a compound according to the present invention contains neither an allyloxycarbonylaminophenyl nor a thiadiazolyl group.

A compound according to the present invention is also more useful as a medicament because of having a high NPY Y1 antagonist activity and/or having a low human P-glycoprotein substrate specificity and being inhibited from elimination from the brain, compared to the compound (A).
non-patent document 1: Nature, vol. 296, p. 659 (1982)
non-patent document 2: International Journal of Obesity, vol. 19, p. 517 (1995)
non-patent document 3: Endocrinology, vol. 133, p. 1753 (1993)
non-patent document 4: British Journal of Pharmacology, vol. 95, p. 419 (1988)
non-patent document 5: Frontiers in Neuroendocrinology, vol. 27, p. 308 (2006)
non-patent document 6: Trends in Pharmacological Sciences, vol. 15, p. 153 (1994)
non-patent document 7: J. Med. Chem., vol. 37, p. 811 (1994)
patent document 1: European Patent No. 355794
patent document 2: Danish Patent No. 3811193
patent document 3: WO94/00486
patent document 4: Japanese Patent Laid-Open No. 6-116284
patent document 5: Japanese Patent Laid-Open No. 6-293794
patent document 6: German Patent DE4301452-A1
patent document 7: WO97/34873

### SUMMARY OF THE INVENTION

In accordance with the present invention, it is desirable to provide a novel compound having a high NPY Y1 antagonist activity and/or a low human P-glycoprotein substrate specificity. In accordance with the present invention, it is also desirable to provide a compound which is a candidate compound for a PET ligand.

As a result of extensive research, the present inventors found a novel heteroarylthiomethyl pyridine compound having a high NPY Y1 antagonist activity and/or a low human P-glycoprotein substrate specificity and the invention was thus accomplished. Specifically, the present invention relates to a compound represented by a formula (I):

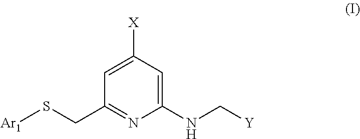

or a pharmaceutically acceptable salt thereof, wherein:
X represents a group selected from a group consisting of:

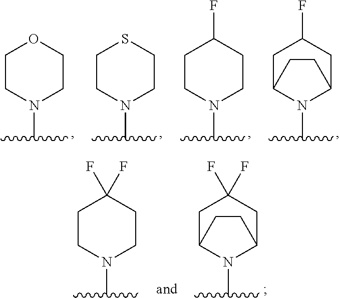

Y represents a group selected from a group consisting of:

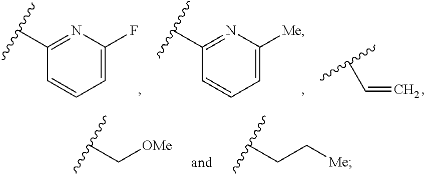

Ar₁ represents a group selected from a group consisting of:

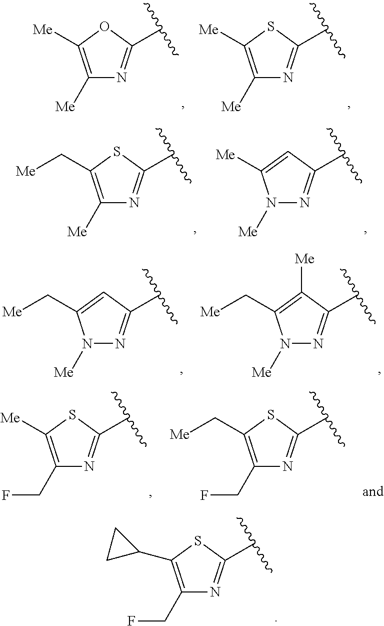

Since the compound (I) according to the present invention has an antagonistic action to NPY, it is useful for treatment and/or prevention of various diseases associated with NPY, for example, cardiovascular diseases such as hypertension, arteriosclerosis, nephropathy, cardiac diseases and angiospasm; diseases of central nervous system such as bulimia, depression, epilepsy, anxiety, alcoholism and dementia; metabolic diseases such as obesity, diabetes mellitus and hormone abnormality, or glaucoma.

The compound (I) according to the present invention also has a low human P-glycoprotein substrate specificity and thus is useful as a medicament.

The compound (I) according to the present invention containing ¹¹C-labeled C or ¹⁸F-labeled F is useful as a PET ligand.

A heteroarylthiomethylpyridine derivative according to the present invention represented by the formula (I), or the pharmaceutically acceptable salt thereof, has a potent antagonistic action to NPY, and it is thus useful for treatment and/or prevention of various diseases associated with NPY, for example, cardiovascular diseases such as hypertension, nephropathy, cardiac diseases and angiospasm; cardiovascular diseases such as hypertension, arteriosclerosis, nephropathy, cardiac diseases and angiospasm; diseases of central nervous system such as bulimia, depression, epilepsy, anxiety, alcoholism and dementia; metabolic diseases such as obesity, diabetes mellitus and hormone abnormality, or glaucoma.

### DETAILED DESCRIPTION OF THE INVENTION

The meanings of terms as used herein are described below, and compounds according to the present invention are further described.

The term “halogen atom” includes, for example, fluorine, chlorine, bromine and iodine atoms.

The term “lower alkyl” means linear or branched C₁₋₆ alkyl and includes, for example, methyl, ethyl, propyl, isopropyl, butyl, isobutyl, sec-butyl, tert-butyl, pentyl, isoamyl, neopentyl, isopentyl, 1,1-dimethylpropyl, 1-methylbutyl, 2-methylbutyl, 1,2-dimethylpropyl, hexyl, isohexyl, 1-methylpentyl, 2-methylpentyl, 3-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1-ethylbutyl, 2-ethylbutyl, 1,2,2-trimethylpropyl and 1-ethyl-2-methylpropyl.

In order to further specifically disclose the compound represented by the formula (I) in accordance with the present invention

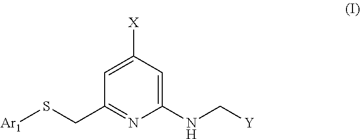

wherein each symbol has the same definition specified above, and
each symbol used in the formula (I) is described referring to specific examples.

X represents a group selected from a group consisting of:

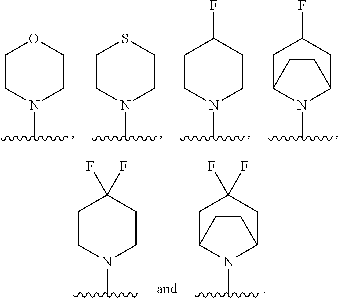

Among these, X is preferably a group selected from

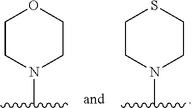

Y represents a group selected from a group consisting of

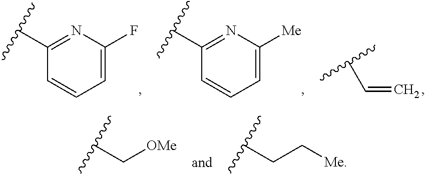

Among these, Y is preferably a group selected from

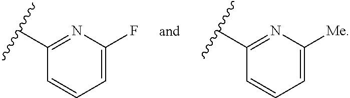

Ar₁ represents a group selected from a group consisting of

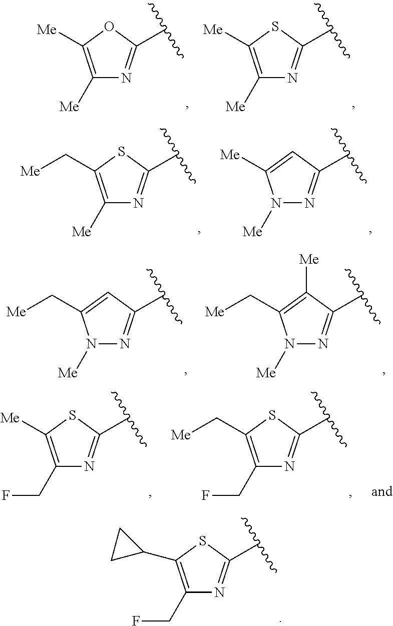

Among these, An is preferably a group selected from

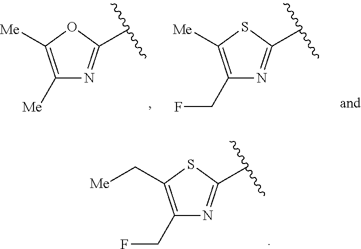

In accordance with a preferred embodiment, any aspects of X, Y and Ar₁ as described above may be combined.

Compounds represented by the formula (I) specifically include, for example,
6-{[(4,5-dimethyl-1,3-oxazol-2-yl)sulfanyl]methyl}-N-[(6-fluoropyridin-2-yl)methyl]-4-(thiomorpholin-4-yl)pyridin-2-amine,
6-{[(4,5-dimethyl-1,3-thiazol-2-yl)sulfanyl]methyl}-N-[(6-methylpyridin-2-yl)methyl]-4-(thiomorpholin-4-yl)pyridin-2-amine,
6-{[(5-ethyl-4-methyl-1,3-thiazol-2-yl)sulfanyl]methyl-4-(4-fluoropiperidin-1-yl)-N-[(6-methylpyridin-2-yl)methyl]pyridin-2-amine,
6-{[(5-ethyl-4-methyl-1,3-thiazol-2-yl)sulfanyl]methyl}-4-[(3-exo)-3-fluoro-8-azabicyclo[3,2,1]octo-8-yl]-N-(prop-2-en-1-yl)pyridin-2-amine,
4-(3,3-difluoro-8-azabicyclo[3,2,1]octo-8-yl)-6-{[(1,5-dimethyl-1H-pyrazol-3-yl)-sulfanyl]methyl}-N-[(6-methylpyridin-2-yl)methyl]pyridin-2-amine,
4-(4,4-difluoropiperidin-1-yl)-6-{[(5-ethyl-1-methyl-1H-pyrazol-3-yl)sulfanyl]methyl}-N-(prop-2-en-1-yl)pyridin-2-amine,
N-butyl-4-(4,4-difluoropiperidin-1-yl)-6-{[(5-ethyl-1,4-dimethyl-1H-pyrazol-3-yl)-sulfanyl]methyl}pyridin-2-amine,
4-(4,4-difluoropiperidin-1-yl)-6-{[(5-ethyl-1,4-dimethyl-1H-pyrazol-3-yl)sulfanyl]-methyl}-N-(2-methoxyethyl)pyridin-2-amine,
6-({[4-(fluoromethyl)-5-methyl-1,3-thiazol-2-yl]sulfanyl}methyl)-N-[(6-methylpyridin-2-yl)methyl]-4-(morpholin-4-yl)pyridin-2-amine,
6-({[5-ethyl-4-(fluoromethyl)-1,3-thiazol-2-yl]sulfanyl methyl)-N-[(6-methylpyridin-2-yl)methyl]-4-(morpholin-4-yl)pyridin-2-amine or
6-({[5-cyclopropyl-4-(fluoromethyl)-1,3-thiazol-2-yl]sulfanyl}methyl)-N-[(6-methyl-pyridin-2-yl)methyl]-4-(morpholin-4-yl)pyridin-2-amine, or the pharmaceutically acceptable salt thereof.

The compounds represented by the formula (I) may be also used as PET ligands by labeling C and F therein with ¹¹C and ¹⁸F, respectively.

The compounds prepared by substituting ¹¹C for C and ¹⁸F for F in the compounds represented by the formula (I) may be produced by processes that are ordinarily used by those skilled in the art or processes similar thereto.

As a PET ligand candidate compound, specifically, for example, a compound prepared by substituting ¹⁸F for F in
6-{[(4,5-dimethyl-1,3-oxazol-2-yl)sulfanyl]methyl}-N-[(6-fluoropyridin-2-yl)methyl]-4-(thiomorpholin-4-yl)pyridin-2-amine,
6-({[4-(fluoromethyl)-5-methyl-1,3-thiazol-2-yl]sulfanyl}methyl)-N-[(6-methylpyridin-2-yl)methyl]-4-(morpholin-4-yl)pyridin-2-amine or
6-({[5-ethyl-4-(fluoromethyl)-1,3-thiazol-2-yl]sulfanyl}methyl)-N-[(6-methylpyridin-2-yl)methyl]-4-(morpholin-4-yl)pyridin-2-amine, among the above-described compounds is preferred.

Also,
6-{[(4,5-dimethyl-1,3-oxazol-2-yl)sulfanyl]methyl}-N-[(6-fluoropyridin-2-yl)methyl]-4-(thiomorpholin-4-yl)pyridin-2-amine,
6-({[4-(fluoromethyl)-5-methyl-1,3-thiazol-2-yl]sulfanyl}methyl)-N-[(6-methylpyridin-2-yl)methyl]-4-(morpholin-4-yl)pyridin-2-amine, and
6-({[5-ethyl-4-(fluoromethyl)-1,3-thiazol-2-yl]sulfanyl}methyl)-N-[(6-methylpyridin-2-yl)methyl]-4-(morpholin-4-yl)pyridin-2-amine are compounds having a notably low human P-glycoprotein substrate specificity.

In accordance with a preferred embodiment of the present invention, the compounds have a high NPY Y1 antagonist activity and a low human P-glycoprotein substrate specificity.

In accordance with another preferred embodiment of the present invention, the compounds have a high NPY Y1 antagonist activity.

In accordance with another preferred embodiment of the present invention, the compounds have a low human P-glycoprotein substrate specificity.

A process for producing the compound according to the present invention will now be described. The compound according to the present invention can be produced by a process illustrated below or processes described in Reference Examples or Examples. However, a process for producing the compound according to the present invention is not limited to such reaction examples.

A compound (I-1) according to the present invention

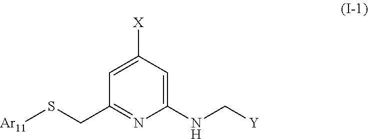

wherein Ar₁₁ is a group represented by

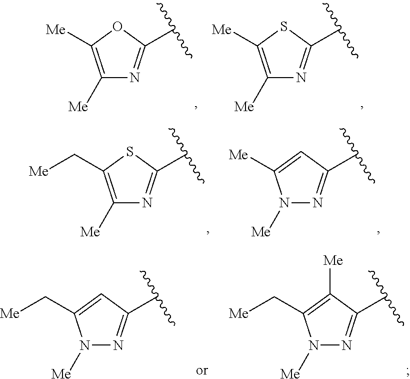

and the other symbols have the same definitions specified above can be produced, for example, by the following process:

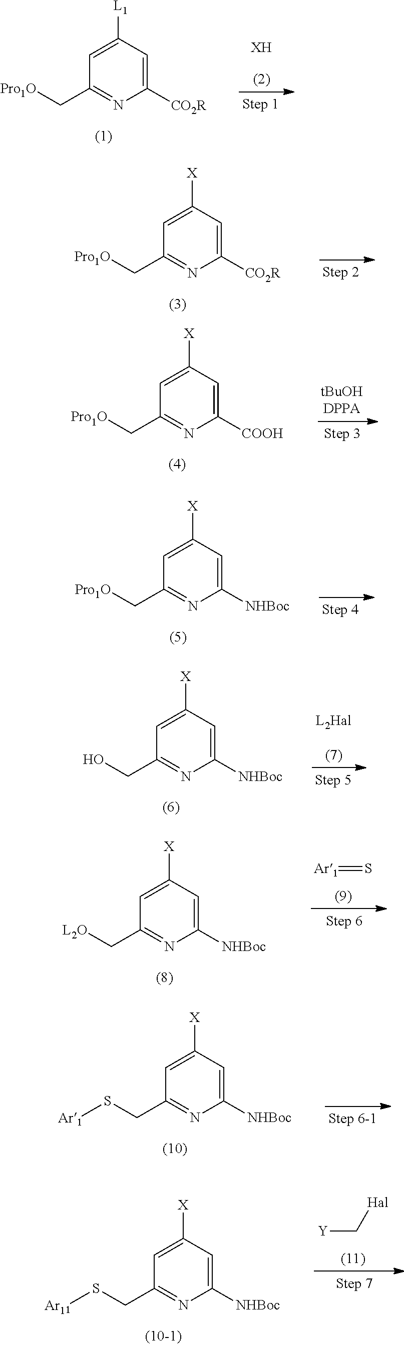

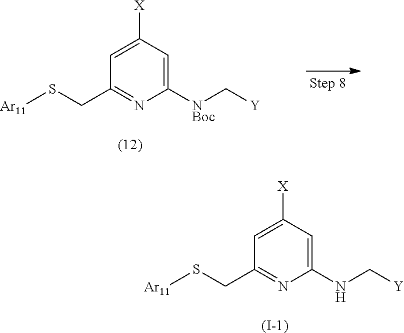

wherein L₁ represents a leaving group such as a trifluoromethanesulfonyloxy group (OTf); R represents a lower alkyl group; Pro₁ represents a protective group for a hydroxy group; Hal represents a halogen atom; L₂ represents methanesulfonyl, trifluoromethanesulfonyl, p-toluenesulfonyl or the like; Ar′₁═S is a compound represented by:

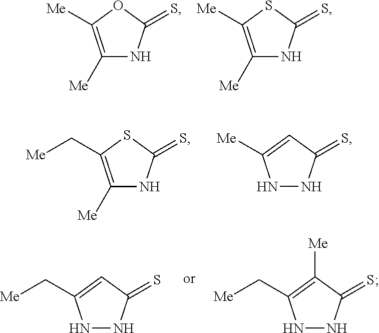

and the other symbols have the same definitions specified above.

### (Step 1)

This step is a process for producing a compound (3) by reacting a compound (1) with a compound (2).

An amount of the compound (2) as used is typically 2.0-20.0 equivalents, preferably 2.0-10.0 equivalents, per 1 equivalent of the compound (1).

This reaction may be also carried out by addition of a base to a reaction system.

Examples of such bases include potassium carbonate, sodium carbonate, cesium carbonate, sodium hydrogencarbonate, potassium hydrogencarbonate, lithium carbonate, trimethylamine, triethylamine and diisopropylethylamine.

An amount of the base is typically 1.0-10.0 equivalents, preferably 1.0-5.0 equivalents, per 1 equivalent of the compound (1).

The compound (1) used in this step may be produced by processes described in Reference Examples, methods equivalent thereto or combinations of these with usual methods.

In addition, the compound (2) XH used in this step means a group represented by

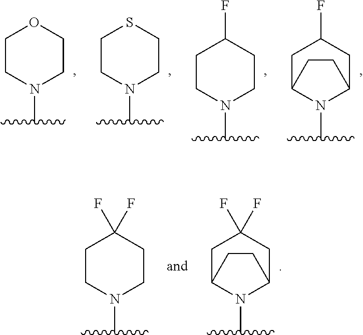

As the compound (2) XH, commercially available compounds or compounds produced by processes described in Reference Examples, methods equivalent thereto or combinations of these with usual methods using commercially available compounds may be used.

The reaction temperature is typically 0-150° C., preferably from room temperature to 100° C.

The reaction time is typically 10 minutes to 48 hours, preferably 10 minutes to 24 hours.

Unless interfering with the reaction, any reaction solvent may be used, examples of which include tetrahydrofuran (sometimes abbreviated as THF), N,N-dimethylformamide (sometimes abbreviated as DMF), N,N-dimethylacetamide (sometimes abbreviated as DMA), dimethylsulfoxide (sometimes abbreviated as DMSO), dimethoxyethane (sometimes abbreviated as DME), toluene, chloroform, methylene chloride and diethyl ether.

The compound (3) thus obtained may be isolated and purified in well-known separation and purification method such as concentration, concentration under reduced pressure, reprecipitation, solvent extraction, crystallization and chromatography, or subjected to the next step without isolation and purification.

### (Step 2)

This step is a process for producing a carboxylic acid compound (4) by hydrolyzing the ester of the compound (3) obtained in the step 1.

Such esters include, for example, methyl ester and ethyl ester.

The reaction in this step can be carried out by a method as described in the document (T.W. Green: Protective Groups in Organic Synthesis, Second Edition, John Wiley & Sons (1991)), methods equivalent thereto or combinations of these with usual methods. For example, the compound (3) can be converted into the compound (4) using sodium hydroxide in methanol.

The compound (4) thus obtained may be isolated and purified in well-known separation and purification method such as concentration, concentration under reduced pressure, reprecipitation, solvent extraction, crystallization and chromatography, or subjected to the next step without isolation and purification.

### (Step 3)

This step is a process for producing a compound (5) by reacting the compound (4) with tert-butanol and diphenylphosphoryl azide in the presence of a base.

Bases as used in this step include, for example, trimethylamine, triethylamine, diisopropylethylamine and pyridine.

An amount of the base is typically 1.0-10.0 equivalents, preferably 1.0-5.0 equivalents, per 1 equivalent of the compound (4).

The above reaction can be carried out in tert-butanol or a mixed solution of tert-butanol and an organic solvent.

Unless interfering with the reaction as the organic solvent, any solvent may be used in this step, examples of which include toluene, THF, 1,4-dioxane and diethyl ether.

An amount of diphenylphosphoryl azide as used is typically 1.0-10.0 equivalents, preferably 1.0-5.0 equivalents, per 1 equivalent of the compound (4).

An amount of tert-butanol as used is typically 1.0 equivalent to greatly excessive amount, per 1 equivalent of the compound (4).

The reaction temperature is typically 0-150° C., preferably from room temperature to 100° C.

The reaction time is typically 10 minutes to 48 hours, preferably 10 minutes to 24 hours.

The compound (5) thus obtained may be isolated and purified in well-known separation and purification method such as concentration, concentration under reduced pressure, reprecipitation, solvent extraction, crystallization and chromatography, or subjected to the next step without isolation and purification.

### (Step 4)

This step is a process for producing a compound (6) by removing a protective group Proₜ for a hydroxy group of the compound (5).

The reaction in this step can be carried out by a method as described in the document (T. W. Green: Protective Groups in Organic Synthesis, Second Edition, John Wiley & Sons (1991)), methods equivalent thereto or combinations of these with usual methods. When a tetrahydropyranyl group is used as Pro₁, the compound (6) can be obtained by reacting the compound (5) with p-toluenesulfonic acid or a hydrate thereof in, for example, ethanol or methanol.

The compound (6) thus obtained may be isolated and purified in well-known separation and purification method such as concentration, concentration under reduced pressure, reprecipitation, solvent extraction, crystallization and chromatography, or subjected to the next step without isolation and purification.

### (Step 5)

This step is a process for producing a compound (8) by reacting the compound (6) with a compound (7) in the presence of a base.

Bases as used in this step include, for example, trimethylamine, triethylamine, diisopropylethylamine, pyridine, potassium carbonate, cesium carbonate, sodium carbonate, sodium hydrogencarbonate, potassium hydrogencarbonate and lithium carbonate.

An amount of the base is typically 1.0-10.0 equivalents, preferably 1.0-5.0 equivalents, per 1 equivalent of the compound (6).

An amount of the compound (7) is typically 1.0-10.0 equivalents, preferably 1.0-5.0 equivalents, per 1 equivalent of the compound (6).

Examples of the compound (7) as used in this step include methanesulfonyl chloride, trifluoromethanesulfonyl chloride, p-toluenesulphonyl chloride and p-nitrobenzenesulfonyl chloride.

The reaction temperature is typically from 0° C. to 100° C., preferably from 0° C. to 50° C.

The reaction time is typically 10 minutes to 48 hours, preferably 10 minutes to 24 hours.

Unless interfering with the reaction, any solvent may be used in this step, examples of which include THF, DMF, chloroform, methylene chloride, ethyl acetate and toluene.

The compound (8) thus obtained may be isolated and purified in well-known separation and purification method such as concentration, concentration under reduced pressure, reprecipitation, solvent extraction, crystallization and chromatography, or subjected to the next step without isolation and purification.

### (Step 6)

This step is a process for producing a compound (10) by reacting the compound (8) with a compound (9) in the presence of a base.

Bases as used include, for example, trimethylamine, triethylamine, diisopropylethylamine, pyridine, diazabicycloundecene, potassium carbonate, cesium carbonate, sodium carbonate, sodium hydrogencarbonate, potassium hydrogencarbonate and lithium carbonate.

An amount of the base is typically 1.0-10.0 equivalents, preferably 1.0-5.0 equivalents, per 1 equivalent of the compound (8).

An amount of the compound (9) is typically 1.0-10.0 equivalents, preferably 1.0-5.0 equivalents, per 1 equivalent of the compound (8).

As the compound (9) or an acceptable salt thereof, commercially available compounds or compounds produced by processes described in Reference Examples, methods equivalent thereto or combinations of these with usual methods using commercially available compounds may be used.

The reaction temperature is typically from 0° C. to 150° C., preferably from 0° C. to 100° C.

The reaction time is typically 10 minutes to 48 hours, preferably 10 minutes to 24 hours.

Unless interfering with the reaction, any solvent may be used in this step, examples of which include THF, DMF, DMSO, chloroform and methylene chloride.

The compound (10) thus obtained may be isolated and purified in well-known separation and purification method such as concentration, concentration under reduced pressure, reprecipitation, solvent extraction, crystallization and chromatography, or subjected to the next step without isolation and purification.

### (Step 6-1)

This step is a process for producing a compound (10-1) by reacting the compound (10) with methyl iodide in a solvent such as DMF in the presence of a base such as potassium carbonate where Ar′₁ is

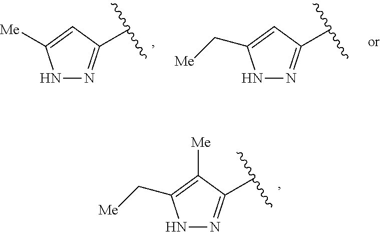

in the compound (10).

Bases as used include potassium carbonate, cesium carbonate, sodium carbonate, sodium hydrogencarbonate, potassium hydrogencarbonate and lithium carbonate.

An amount of the base is typically 1.0-10.0 equivalents, preferably 1.0-5.0 equivalents, per 1 equivalent of the compound (10).

An amount of methyl iodide as used is typically 1.0-10.0 equivalents, preferably 1.0-5.0 equivalents, per 1 equivalent of the compound (10).

The reaction temperature is typically from 0° C. to 150° C., preferably from 0° C. to 100° C.

The reaction time is typically 10 minutes to 48 hours, preferably 10 minutes to 24 hours.

Solvents as used include DMF, THF, DMSO, chloroform and methylene chloride.

The compound (10-1) thus obtained may be isolated and purified in well-known separation and purification method such as concentration, concentration under reduced pressure, reprecipitation, solvent extraction, crystallization and chromatography, or t subjected to the next step without isolation and purification.

### (Step 7)

This step is a process for producing a compound (12) by reacting the compound (10) or (10-1) with a compound (11):

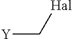

in the presence of a base.

Hal is preferably a chlorine or bromine atom.

Bases as used in this step include sodium hydride, potassium hydride and potassium tert-butoxide.

An amount of the base is typically 1.0-10.0 equivalents, preferably 1.0-5.0 equivalents, per 1 equivalent of the compound (10) or (10-1).

An amount of the compound (11) as used is typically 1.0-5.0 equivalents, preferably 1.0-3.0 equivalents, per 1 equivalent of the compound (10) or (10-1).

The reaction temperature is typically from 0° C. to 150° C., preferably from 0° C. to 100° C.

The reaction time is typically 10 minutes to 48 hours, preferably 10 minutes to 24 hours.

Unless interfering with the reaction, any solvent may be used in this step, examples of which include THF, DMF, DMSO, chloroform and methylene chloride.

The compound (12) thus obtained may be isolated and purified in well-known separation and purification method such as concentration, concentration under reduced pressure, reprecipitation, solvent extraction, crystallization and chromatography, or subjected to the next step without isolation and purification.

### (Step 8)

This step is a process for producing a compound (I-1) according to the present invention by removing a Boc group of the compound (12).

The reaction in this step can be carried out by a method as described in the document (T. W. Green: Protective Groups in Organic Synthesis, Second Edition, John Wiley & Sons (1991)), methods equivalent thereto or combinations of these with usual methods.

The compound (I-1) thus obtained may be isolated and purified in well-known separation and purification method such as concentration, concentration under reduced pressure, reprecipitation, solvent extraction, crystallization and chromatography.

In addition, a compound (I-2) according to the present invention

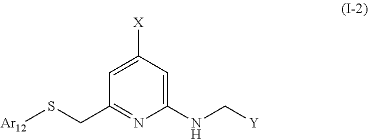

wherein Ar₁₂ is a group represented by

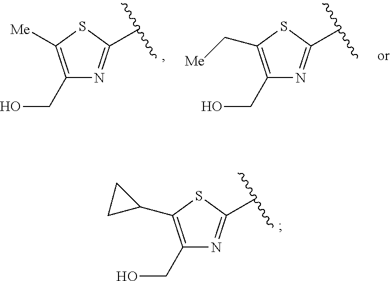

and the other symbols have the same definitions specified above, can be produced, for example, by the following process:

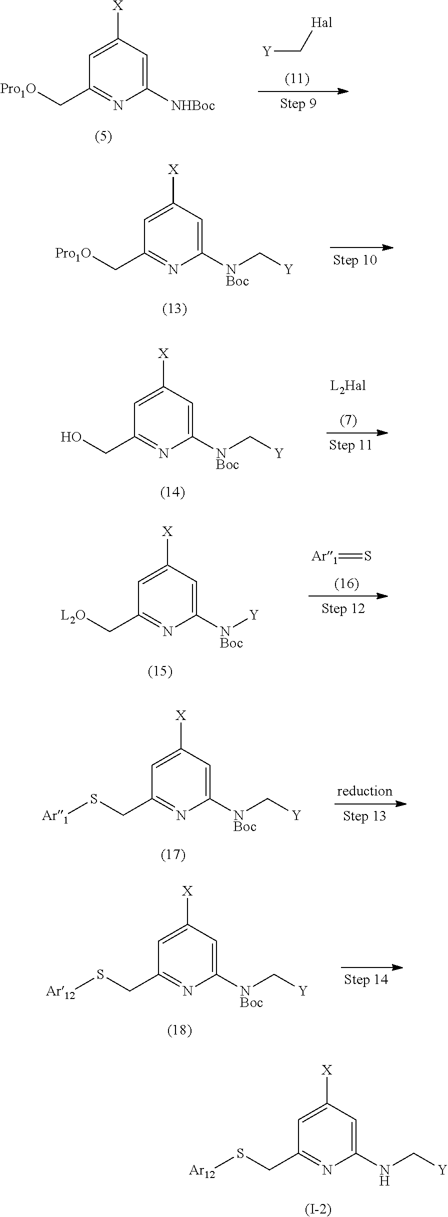

wherein Ar″₁═S means a compound represented by

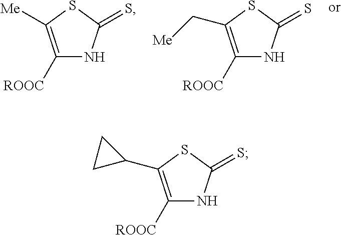

Ar′₁₂ is a group represented by

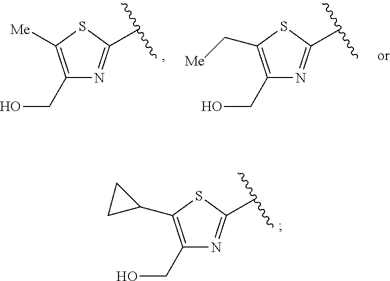

and the other symbols have the same definitions specified above.

### (Step 9)

This step is a process for producing a compound (13) by reacting the compound (5) with the compound (11)

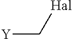

in the presence of a base.

The reaction in this step can be carried out by the same method as in Step 7 described above, methods equivalent thereto or combinations of these with usual methods.

The compound (13) thus obtained may be isolated and purified in well-known separation and purification method such as concentration, concentration under reduced pressure, reprecipitation, solvent extraction, crystallization and chromatography, or subjected to the next step without isolation and purification.

### (Step 10)

This step is a process for producing a compound (14) by removing a protective group Pro₁ for a hydroxy group of the compound (13).

The reaction in this step can be carried out by the same method as in Step 4 described above, methods equivalent thereto or combinations of these with usual methods.

The compound (14) thus obtained may be isolated and purified in well-known separation and purification method such as concentration, concentration under reduced pressure, reprecipitation, solvent extraction, crystallization and chromatography, subjected to the next step without isolation and purification.

### (Step 11)

This step is a process for producing a compound (15) by reacting the compound (14) with the compound (7) in the presence of a base.

The reaction in this step can be carried out by the same method as in Step 5 described above, methods equivalent thereto or combinations of these with usual methods.

The compound (15) thus obtained may be isolated and purified in well-known separation and purification method such as concentration, concentration under reduced pressure, reprecipitation, solvent extraction, crystallization and chromatography, or subjected to the next step without isolation and purification.

### (Step 12)

This step is a process for producing a compound (17) by reacting the compound (15) with a compound (16) in the presence of a base.

The reaction in this step can be carried out by the same method as in Step 6 described above, methods equivalent thereto or combinations of these with usual methods.

The compound (17) thus obtained may be isolated and purified in well-known separation and purification method such as concentration, concentration under reduced pressure, reprecipitation, solvent extraction, crystallization and chromatography, or subjected to the next step without isolation and purification.

### (Step 13)

This step is a process for producing a compound (18) by reducing an ester group COOR of the compound (17).

Reducing agents as used in this step include, for example, lithium aluminum hydride, diisobutylaluminum hydride, lithium borohydride, sodium borohydride, borane-tetrahydrofuran complex and borane-methylsulfide complex.

An amount of the reducing agent is typically 1.0-10.0 equivalents, preferably 1.0-5.0 equivalents, per 1 equivalent of the compound (17).

The reaction time is typically 10 minutes to 48 hours, preferably 10 minutes to 24 hours.

The reaction temperature is typically from −100° C. to 150° C., preferably from −78° C. to 100° C.

Unless interfering with the reaction, any solvent may be used in this step, examples of which include THF, toluene, diethyl ether, hexane, methanol, ethanol, chloroform and methylene chloride.

The compound (18) thus obtained may be isolated and purified in well-known separation and purification method such as concentration, concentration under reduced pressure, reprecipitation, solvent extraction, crystallization and chromatography, or subjected to the next step without isolation and purification.

### (Step 14)

This step is a process for producing a compound (1-2) according to the present invention by removing a Boc group of the compound (18) and then performing treatment with DAST (diethylaminosulfur trifluoride).

The reaction of removing a Boc group in this step can be carried out by the method as described in the document (T. W. Green: Protective Groups in Organic Synthesis, Second Edition, John

Wiley & Sons (1991)), methods equivalent thereto or combinations of these with usual methods.

The compound according to the present invention is obtained by fluorinating a hydroxy group by removing a Boc group and then performing treatment with DAST.

An amount of DAST as used is typically 1.0-10.0 equivalents, preferably 1.0-5.0 equivalents, per 1 equivalent of the compound in which a Boc group of the compound (18) is removed.

The reaction time is typically 10 minutes to 48 hours, preferably 10 minutes to 24 hours.

The reaction temperature is typically from −100° C. to 100° C., preferably from −78° C. to 50° C.

Unless interfering with the reaction, any solvent may be used in this step, examples of which include THF, chloroform and methylene chloride.

The compound (I-2) thus obtained may be isolated and purified in well-known separation and purification method such as concentration, concentration under reduced pressure, reprecipitation, solvent extraction, crystallization and chromatography.

Heteroarylthiomethylpyridine derivatives provided by the present invention may be present as the pharmaceutically acceptable salts, which can be produced according to usual methods using the compound (I) according to the present invention and a compound represented by the formula (I-1) or (I-2) encompassed by the compound (I).

Specifically, when the compound according to the formula (I) has a basic group derived from, for example, an amino or pyridyl group, in a molecule, the compound can be also converted into the corresponding pharmaceutically acceptable salt by treating the compound with an acid.

Examples of such acid addition salts include hydrohalic acid salts such as hydrochloride, hydrofluorate, hydrobromide and hydroiodide; inorganic acid salts such as nitride, perchlorate, sulfate, phosphate and carbonate; lower alkyl sulfonate salts such as methanesulfonate, trifluoromethanesulfonate and ethanesulfonate; aryl sulfonates such as benzensuplhonate and p-toluenesulfonate; organic salts such as fumarate, succinate, citrate, tartrate, oxalate and maleate; and acid addition salts of organic acids, for example, amino acids, such as glutamate and aspartate. In addition, when the compound according to the present invention has an acidic group, such as carboxyl, in the group, the compound can be also converted into a corresponding pharmaceutically acceptable salt by processing the compound with a base. Examples of such base addition salts include alkali metal salts such as sodium and potassium; alkaline earth metal salts such as calcium and magnesium; ammonium salts; and salts of organic bases such as guanidine, triethylamine and dicyclohexylamine. Furthermore, the compound according to the present invention may be present in the form of a free compound or any hydrate or solvate of a salt thereof.

Furthermore, in the compound according, to the present invention, a stereoisomer or a tautomer, such as an optical isomer, a diastereoisomer or a geometrical isomer, is sometimes present depending on the form of a substituent. It will be appreciated that these isomers are encompassed entirely by compounds according to the present invention. Furthermore, it will be appreciated that any mixture of these isomers is also encompassed by compounds according to the present invention.

The utility of compounds according to the present invention as a medicament is specifically proved, for example, in the following pharmacological examples 1 or 2.

### Pharmacological Test Example 1

### NPY Binding Inhibition Test

A cDNA sequence encoding a human NPY Y1 receptor (Accession No. L07615) was cloned into expression vectors pEF1x (made by Invitrogen Inc.). The obtained expression vectors were transfected to host cells CHO-K1 NFAT β-Lactamase (Aurora) by cationic lipid method [see Proceedings of the National Academy of Sciences of the United States of America, 84: 7413 (1987)] to give NPY Y1 receptor expression cells.

A membrane sample prepared from the cells which expressed the NPY Y1 receptor was incubated together with a test compound and [¹²⁵I] peptide YY (manufactured by Amersham) (20,000 cpm) in an assay buffer (HEPES buffer (pH 7.4) containing 20 mM HEPES, 0.5% BSA, 1 mM phenylmethylsulfonylfluoride and 0.1% bacitracin) at 25° C. for 2 hours, then filtered through a glass filter GF/C, and washed with 20 mM HEPES buffer (pH 7.4), followed by measuring the radioactivity of the cake on the glass filter. Nonspecific binding was measured in the presence of 1 μM peptide YY and a 50% Inhibitory Concentration (IC₅₀ value) of the test compound against specific [¹²⁵I] peptide YY binding was determined [Endocrinology, 131: 2090 (1992)].

The results are summarized in Table 1.

| Test Compound | IC50 (nM) |
|---|---|
| Example 1 | 0.27 |
| Example 2 | 0.17 |
| Example 3 | 0.26 |
| Example 4 | 0.27 |
| Example 5 | 0.24 |
| Example 6 | 0.28 |
| Example 7 | 0.27 |
| Example 8 | 0.27 |
| Example 9 | 0.20 |
| Example 10 | 0.13 |
| Example 11 | 0.19 |

In addition, the IC₅₀ value of the compound (A) according to WO97/34873 was 0.33 (nM).

As shown above, the compounds in accordance with the present invention potently inhibited peptide YY (NPY homologue) binding to NPY Y1 receptors.

Based on the results, the compound (I) according to the present invention is useful as an agent for prevention and/or treatment of various diseases associated with NPY, for example, cardiovascular diseases such as hypertension, nephropathy, cardiac diseases and angiospasm; diseases of central nervous system such as bulimia, depression, epilepsy and dementia; metabolic diseases such as obesity, diabetes mellitus and hormone abnormality, or glaucoma, particularly, for example, as an agent for prevention and/or treatment of bulimia, obesity or diabetes mellitus.

### Pharmacological Test Example 2

### Assay on P-Glycoprotein Substrate Specificity

P-glycoprotein (P-gp) substrate specificities were assessed in a transcellular transport assay system using cells expressing human P-glycoprotein. The human P-glycoprotein expression cells employing porcine kidney-derived LLC-PK1 cells as host cells were obtained from Dr. Alfred Schinkel in the Netherlands Cancer Institute [see Journal of Clinical Investigation, vol. 96, p. 1698 (1995)]. The cells were seeded on a filter membrane of 24-well HTS multiwell insert system (manufactured by Beckton Dickinson) to have a concentration of 0.15 million cells/well. The cells were cultured for about 1 week to form a single layer film by the cells. The multiwell insert system is divided into two compartments (the apical membrane side compartment and the basolateral membrane side compartment). Prior to the transport experiment, cell culture media on both sides (apical and basolateral sides) were replaced by Hank's balanced salt solution (HBSS) containing an assay buffer solution (10 mM Hepes (pH 7.4)). After about 1 hour, the basolateral and apical solutions were replaced by an assay buffer solution (0.5 mL) and a 1 μM test compound-containing assay buffer solution (0.5 mL), respectively, and the transport experiment was started for transportation from Apical to Basolateral (from A to B). After 3 hours, each 50 μL was collected as a measuring sample from both of the apical and basolateral sides. The reverse operation thereof was carried out for transportation from Basolateral to Apical (from B to A). A test compound addition side was “donor”, and the other side was “receiver.” The formation of the cell single layer film was confirmed by previously adding 0.5 μM Dextran Texas Red (3000 MW) together with the test compound to the donor side and terminating incubation, followed by measuring a leakage level to the receiver side with a fluorescence plate reader (Ex 590 nm to Em 635 nm). After termination of the transport experiment, an equal amount of acetonitrile containing the internal standard was added to each measuring sample. The measuring samples were measured using LC-MS/MS to calculate the concentrations of the test compounds by a relative calibration curve method. The permeability coefficients of the test compounds transported in each of from A to B and from B to A were calculated (Papp: Expression 1). The ratios of the permeability coefficients between A to B and B to A were also determined (BA/AB: Expression 2). BA/AB was considered to be an index of P-glycoprotein (p-gp) substrate specificity [see Drug Metabolism and Disposition, vol. 31, p. 1251, (2003)]. The results are summarized in Table 2.

| Test Compound | BA/AB |
|---|---|
| Example 1 | 1.9 |
| Example 9 | 1.8 |
| Example 10 | 1.7 |
| Compound (A) | 5.0 |

In the above assay on human P-glycoprotein, BA/AB of <3 is preferred.

Papp=(0.5 mL×CR)/((CD+CR)×3 hr×Area) Expression 1

BA/AB=Papp(B to A)/Paap(A to B) Expression 2

Area: surface area of filter; CD: concentration of test compound in donor side after 3 hours; CR: concentration of test compound in receiver side after 3 hours

The compound (A) used in the pharmacology tests 1 and 2 are the compound according to WO97/34873.

Compounds represented by the formula (I) may be orally or parenterally administered and can be formulated in forms suitable for such administration, resulting in possible provision of the compounds as treatment agents for cardiovascular diseases such as angina pectoris, acute congestive heart failure, myocardial infarction, hypertension, nephropathy, electrolyte abnormality, angiospasm and arteriosclerosis; diseases of central nervous system such as bulimia, depression, anxiety, convulsion, epilepsy, dementia, pain, alcoholism, withdrawal symptoms associated with drug deprivation, circadian rhythm abnormality, schizophrenia, memory disorder, sleep disorder and cognition disorder; metabolic diseases such as obesity, diabetes mellitus, abnormal hormone secretion, hypercholesterolemia, hyperlipidemia, gout and fatty liver; reproductive system diseases such as infertility, premature labor and sexual function disorder; gastrointestinal system diseases; respiratory system diseases; inflammatory diseases; glaucoma; for example, atherosclerosis, hypogonadism, hyperandrogenism, polycystic ovary syndrome, hypertrichosis, gastrointestinal motility disorder, gastroesophageal reflux associated with obesity, obesity hypoventilation syndrome (Pickwickian syndrome), sleep apnea syndrome, inflammation, systemic vasculitis, degenerative arthritis, insulin resistance, bronchoconstriction, alcoholophilia, metabolic syndrome (syndrome X), Alzheimer's disease, cardiac hypertrophy, left ventricular hypertrophy, hypertriglyceridemia and low HDL cholesteremia; cardiovascular diseases such as coronary heart disease (CHD), cerebrovascular disease, stroke, peripheral vascular disease and sudden death; gallbladder disease; cancer (breast cancer, endometrial cancer or colon cancer); breathlessness; hyperuricemia; fertility disorder; lumbago; or anesthetic allergy. When a compound of the present invention is clinically used, a pharmaceutically acceptable additive may be also added, depending on its dosage form, to produce various preparations, followed by administering the preparations. Such additives, for which various additives that are used typically in the field of preparation can be used, include, for example, gelatin, lactose, saccharose, titanium oxide, starch, crystalline cellulose, hydroxypropyl methylcellulose, carboxymethyl cellulose, corn starch, microcrystalline wax, white petrolatum, magnesium aluminometasilicate, anhydrous calcium phosphate, citric acid, trisodium citrate, hydroxypropylcellulose, sorbitol, sorbitan fatty acid ester, polysorbates, sucrose fatty acid esters, polyoxyethylene, hydrogenated castor oil, polyvinyl pyrrolidone, magnesium stearate, light anhydrous silicic acid, talc, vegetable oil, benzyl alcohol, gum arabic, propylene glycol, polyalkylene glycol, cyclodextrin and hydroxypropyl cyclodextrin.

Examples of dosage forms as formulated mixtures with such additives include solid preparations such as tablets, capsules, granules, powder and suppositories; and liquid preparations such as syrups, elixirs and injectables. Such preparations may be formulated according to the techniques well-known in the art of pharmaceutical formulation. Liquid preparations may be in the form of preparations which are dissolved or suspended in water or other appropriate media just before use. In the case of injectable preparations in particular, they may be dissolved or suspended in physiological saline or glucose solution if necessary, optionally together with a buffer and a preservative.

When compounds of the present invention are used clinically, for example, a daily dose for an adult is 0.01-100 mg/kg, preferably 0.03-1 mg/kg in a single dose or in divided doses when administered orally, or 0.001-10 mg/kg, preferably 0.001-0.1 mg/kg, more preferably 0.01-0.1 mg/kg, in a single dose or in divided doses when administered parenterally, though the dose and the frequency of dosage may vary depending upon the sex, age, body weight, the severity of condition of a patient, and the type and range of the desired therapeutic effects.

An ordinarily skilled physician, veterinarian or clinician can readily determine and prescribe the effective amount of the drug required to prevent, suppress or arrest the progress of diseases.

Such preparations may contain a compound of the present invention at a rate of 1.0-100%, preferably 1.0-60%, by weight of the total drug. Such preparations may also contain other therapeutically-effective compounds.

The compounds of the present invention can be used in combination with other agents useful for treating metabolic and/or eating disorders. The individual components of such combinations can be administered separately at different times during the course of therapy or concurrently in divided or single preparations. The present invention is therefore to be understood as embracing all such regimes of simultaneous or alternating treatment and the term “administering” herein is to be interpreted accordingly. It will be understood that the scope of combinations of the compounds of this invention with other agents useful for treating metabolic and/or eating disorders includes in principle any combination with any pharmaceutical composition useful for treating metabolic and/or eating disorders.

Diabetes mellitus is caused by multiple factors and is characterized by elevated levels of plasma glucose (hyperglycemia) in the fasting state. There are two generally recognized forms of diabetes mellitus: type 1 diabetes mellitus, or insulin dependent diabetes mellitus (IDDM) caused by hyposecretion of insulin which is a hormone regulating glucose utilization, and type 2 diabetes mellitus, or non-insulin dependent diabetes mellitus (NIDDM), wherein patients exhibit hyperinsulinemia (plasma insulin levels that are similar or even elevated in comparison with non-diabetic subjects), while at the same time demonstrating hyperglycemia. Type 1 diabetes mellitus is typically treated with exogenous insulin administered via injection. However, type 2 diabetes mellitus often exhibits the phenomena of aggravating insulin resistance, such that the effect of insulin in stimulating glucose and lipid metabolism in the main insulin-sensitive tissues, namely, muscle, liver and adipose tissues, is diminished. In patients with non-insulin dependent diabetes mellitus (NIDDM), the plasma insulin levels, even when they are elevated, are insufficient to overcome the pronounced insulin resistance, resulting in hyperglycemia. Therefore, the treatment with single administration of exogenous insulin becomes difficult.

Insulin resistance is not yet completely understood. Resistance to insulin results in insufficient activation of glucose uptake, diminished oxidation of glucose and storage of glycogen in muscle, inadequate repression of lipolysis in adipose tissue and inadequate glucose production and secretion by the liver. The persistent or uncontrolled hyperglycemia that occurs in diabetes mellitus is associated with increased morbidity and mortality. Type 2 diabetes mellitus is at increased risk of developing cardiovascular complications, for example, atherosclerosis, coronary heart disease, stroke, peripheral vascular disease, hypertension, nephropathy, neuropathy and retinopathy.

Non-insulin dependent diabetes is also associated with cardiac hypertrophy, in particular left ventricular hypertrophy (Devereux, R. B., Circulation, 101:2271-2276 (2000)). Cardiac hypertrophy, such as left ventricular hypertrophy, is due to the response of the heart to chronic pressure or intravascular volume overload. Left ventricular hypertrophy (LVH) is characterized by thickening of the left ventricular wall, including increased left ventricular mass and increased left ventricular wall thickness, and is defined as a left ventricular mass index exceeding 131 g/m² of the body surface area in men, and 100 g/m² in women (Savage et al., The Framingham Study, Circulation, 75 (1 Pt 2): 26-33 (1987)).

Left ventricular hypertrophy is associated with increased incidence of cardiovascular diseases, such as congestive heart failure, ischaemic heart disease, cardiovascular and all-cause mortality, sudden death and stroke. Therefore, regression of left ventricular hypertrophy is associated with a reduction in cardiovascular risk. The incidence of morbid events in patients with progression of left ventricular hypertrophy has been reported to be greater than that in patients with regression of left ventricular hypertrophy.

Current treatments for hypertrophy include non-pharmacological interventions, such as weight reduction, sodium restriction and aerobic physical exercise, can reduce left ventricular mass (Ghali, J. K. et al., American Journal of Geriatric Cardiology, 6:38-49 (1997)).

Many patients who have insulin resistance but have not yet developed type 2 diabetes mellitus are also at a risk of developing metabolic syndrome, also referred to as syndrome X or pluri-metabolic syndrome. The period of 5 to 10 years preceding the development of impaired glucose tolerance is associated with a number of hormonal imbalances, which give rise to an enlargement of visceral fat mass, hypertension, insulin resistance and hyperlipidemia (Bjornstop, P., Current Topics in Diabetes Research, eds. Belfore, F., Bergman, R. N., and Molinath, G. M., Front Diabetes, Basel, Karger, 12:182-192 (1993)). Similarly, metabolic syndrome is characterized by insulin resistance, along with enlargement of visceral fat mass, hyperinsulinemia, hyperglycemia, syndrome X, low HDL and high VLDL. Although the causal relationship between the various components of metabolic syndrome remains to be confirmed, insulin resistance is likely to play an important role (Requen, G M., et al., N. Eng. J. Med. 334:374-381 (1996); Despres, J-P., et al., N. Engl. J. Med. 334:952-957 (1996); Wajchenberg, B. L., et al., Diabetes/Metabolism Rev. 10:19-29 (1994)). Metabolic syndrome patients, whether or not they develop diabetes mellitus, are at increased risk of developing the cardiovascular complications listed above. Associations have been recently reported to be also found between left ventricular hypertrophy and metabolic syndrome (Marcus, R. et al. Circulation, 90:928-936 (1994); Lind, L. et al., J Hypertens. 13:433-38 (1995); Paolisso, G et al., Am J Hypertens., 10:1250-1256 (1997)).

Type 2 diabetes mellitus is treated with a variety of therapeutic agents including PPAR agonists such as glitazones; biguanides; protein tyrosine phosphatase-1B inhibitors; dipeptidyl peptidase IV inhibitors; insulin; insulin mimetics; sulfonylureas; meglitinides; α-glucoside hydrolase inhibitors; and α-amylase inhibitors.

Increasing the plasma level of insulin by administration of sulfonylureas (for example tolbutamide and glipizide) or meglitinides, which stimulate the pancreatic β-cells to secrete more insulin, and by injection of insulin when sulfonylureas or meglitinides become ineffective, can result in insulin concentrations high enough to stimulate insulin-resistant tissues. However, hypoglycemia, dangerously low levels of plasma glucose, can result, and increasing insulin resistance due to the even higher plasma insulin levels can occur. The biguanides increase insulin sensitivity resulting in some correction of hyperglycemia. Alpha-amylase inhibitors inhibit the enzymatic degradation of starch or glycogen into maltose, have the action of delaying absorption of glucose in the intestine, and also reduces the amounts of bioavailable sugars. Metformin monotherapy is often used for treating type 2 diabetes mellitus patients who also develop obesity and/or dyslipidemia. Lack of appropriate response to metformin will be followed by treatment with sulfonylureas, thiazolidinediones, insulin or alpha glucosidase inhibitors. However, the two biguanides, phenformin and metformin, can also induce lactic acidosis and nausea/diarrhea, respectively. Alpha glucosidase inhibitors, such as acarbose, causes intestinal functional disorder.

The glitazones, also known as thiazolidinediones (such as 5-benzylthiazolidine-2,4-diones), are a more recently described class of compounds with potential for a novel mode of action in ameliorating many symptoms of type 2 diabetes mellitus. These agents, which are agonists of the peroxisome proliferator activated receptor (PPAR) gamma subtype, substantially increase insulin sensitivity in muscle, liver and adipose tissue in several animal models of type 2 diabetes mellitus, resulting in partial or complete correction of the elevated plasma levels of glucose without inducing hypoglycemia. Newer PPAR agonists that are being developed for treatment of type 2 diabetes mellitus and/or dyslipidemia are agonists of one or more of the PPAR alpha, gamma and delta subtypes.

However, treatment of diabetes mellitus with PPARγ agonists sometimes results in cardiac hypertrophy, or an increase in heart weight. Recent labeling revisions for Avandia (rosiglitazone maleate), a PPARγ agonist, suggest that patients may experience fluid retention and volume-related events, such as edema and congestive heart failure. Cardiac hypertrophy related to PPARγ agonist treatment is likely to be typically treated by withdrawing PPAR treatment.

Treatment of type 2 diabetes mellitus also typically includes physical exercise, weight control and dieting. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Furthermore, weight reduction by increased exercise is difficult for most patients with diabetes mellitus because the patients may also develop related symptoms.

Abnormal glucose homeostasis is also associated directly or indirectly with obesity, hypertension and lipidosis. Obesity also increases the likelihood of insulin resistance, and increases the likelihood that the resulting insulin resistance will increase with increasing body weight. Therefore, therapeutic control of glucose homeostasis, lipid metabolism, obesity and hypertension are critically important in the clinical management and treatment of diabetes mellitus.

Obesity, which can be defined as a body weight more than 20% above the ideal body weight, is a major health concern in Western societies. It is estimated that one out of three adults in the United States is overweight or obese. Obesity is the result of a positive energy balance, as a consequence of increased ratio of caloric intake to energy expenditure. [B. Staels et al., J. Biol. Chem. 270 (27), 15958 (1995); F. Lonnquist et al., Nature Medicine 1 (9), 950 (1995)]. Although the molecular factors regulating food intake and body weight balance are incompletely understood, several genetic factors have been identified.

Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include: type 2 diabetes mellitus; hypertension; hyperinsulinism; insulin resistance; lipidosis; hyperlipidemia; endometrial, breast, prostate, kidney and large intestine cancers; degenerative arthritis; respiratory complications, such as non-obstructive sleep apnea syndrome; gallstone disease; arteriosclerosis; cardiac disease; abnormal heart rhythms; and arrhythmia (Kopelman, P. G., Nature 404, 635-643 (2000)). Obesity is also associated with metabolic syndrome, circulatory disorder such as cardiac hypertrophy, in particular left ventricular hypertrophy, premature death, a significant increase in mortality and morbidity from stroke, myocardial infarction, congestive heart failure, coronary heart disease and sudden death.

Abdominal obesity has been linked with a high risk of coronary artery disease, and with three of its major risk factors: high blood pressure, diabetes mellitus that starts in adulthood, and hyperlipidemia. Losing weight dramatically reduces these risks. Abdominal obesity is further closely associated with abnormal glucose tolerance, hyperinsulinemia, hypertriglyceridemia, and other disorders associated with metabolic syndrome (syndrome X), such as decreased levels of high density lipoproteins (HDL) and increased levels of very low density lipoproteins (VLDL) (Montague et al., Diabetes, 2000, 49:883-888).

Obesity and obesity-related diseases, such as diabetes mellitus, are often treated by encouraging patients to lose weight by reducing their food intake or by increasing their exercise level, thereby increasing their energy output. A sustained weight loss of 5% to 10% of body weight can lead to improvement of obesity-related diseases such as diabetes mellitus, left ventricular hypertrophy, degenerative arthritis and cardiorespiratory dysfunction.

Weight loss drugs used for the treatment of obesity include orlistat (Davidson, M. H. et al. (1999) JAMA 281:235-42), dexfenfluramine (Guy Grand, B. et al. (1989) Lancet 2:1142-5), sibutramine (Bray, G. A. et al. (1999) Obes. Res. &:189-98] and phentermine (Douglas, A. et al. (1983) Int. J. Obes. 7:591-5). However, the side effects of such anti-obesity agents may limit their use. Dexfenfluramine was withdrawn from the market because of suspected valvular heart disease; orlistat is limited by gastrointestinal side effects; and the use of sibutramine is limited by its cardiovascular side effects which have led to reports of deaths and its withdrawal from the market in Italy.

The term “diabetes mellitus,” as used herein, includes both insulin dependent diabetes mellitus (i.e., IDDM, also known as type 1 diabetes mellitus) and non-insulin dependent diabetes mellitus (i.e., NIDDM, also known as type 2 diabetes mellitus). The compositions of the present invention are useful for treating both type 1 and type 2 diabetes. The compositions are especially effective for treating type 2 diabetes mellitus. The compositions of the present invention are also useful especially for treating and/or preventing gestational diabetes mellitus.

Compounds or combination compositions of the present invention are efficacious for treatment of diabetes mellitus. One outcome of the treatment may be decreasing the glucose level in a subject with elevated glucose levels. Another outcome of the treatment may be decreasing insulin levels in a subject with elevated insulin levels. Another outcome of the treatment is decreasing plasma triglycerides in a subject with elevated plasma triglycerides. Another outcome of the treatment is decreasing LDL cholesterol in a subject with high LDL cholesterol levels. Another outcome of the treatment is increasing HDL cholesterol in a subject with low HDL cholesterol levels. Another outcome of the treatment is increasing insulin sensitivity. Another outcome of the treatment may be enhancing glucose tolerance in a subject with abnormal glucose tolerance. Another outcome of the treatment may be decreasing insulin resistance.

Compounds or combination compositions of the present invention are efficacious for prevention of diabetes mellitus.

The term “hypertension” as used herein includes essential hypertension wherein the cause is not known or where hypertension is due to at least one cause, such as changes in both the heart and blood vessels; and secondary hypertension wherein the cause is known. Causes of secondary hypertension include, but are not limited to, obesity; kidney disease; hormonal disorders; use of certain drugs, such as oral contraceptives, adrenocorticosteroids, cyclosporines, and the like. The term “hypertension” encompasses high blood pressure, in which both the systolic and diastolic pressure levels are elevated, and isolated systolic hypertension, in which only the systolic pressure is elevated to greater than or equal to 140 mm Hg, while the diastolic pressure is less than 90 mm Hg. One outcome of treatment is decreasing blood pressure in a subject with high blood pressure.

Lipidosis or disorders of lipid metabolism, include various conditions characterized by abnormal concentrations of one or more lipids (for example, cholesterol and triglycerides), and/or apolipoproteins (for example, apolipoproteins A, B, C and E), and/or lipoproteins (for example, macromolecular complexes formed by the lipid and the apolipoprotein that allow lipids to circulate in blood, such as LDL, VLDL and IDL). Hyperlipidemia is associated with abnormally high levels of lipids, LDL and VLDL cholesterol, and/or triglycerides.

The term “metabolic syndrome,” also known as syndrome X, is denned in the Third Report of the National Cholesterol Education Program Expert Panel on Detection, Evaluation and Treatment of High Blood Cholesterol in Adults (ATP-III) (E. S. Ford et al., JAMA, vol. 287 (3), Jan. 16, 2002, pp 356-359). Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “left ventricular hypertrophy” (LVH) as used herein includes three patterns of left ventricular hypertrophy that have been identified based on left ventricular mass index (LVMI=left ventricular mass (g) divided by body surface area (m²)) and relative wall thickness (RWT=2× posterior wall thickness/left ventricular end diastolic diameter). The three patterns means: concentric LVH which is typically exemplified by a left ventricular mass index of 144 and a relative wall thickness of 0.52; eccentric LVH which is typically exemplified by a left ventricular mass index of 136 and a relative wall thickness of 0.38; and concentric left ventricular remodeling which is typically exemplified by a LVMI of 93 and a relative wall thickness of 0.38. Normal LVMI is typically 85, and normal RWT is typically about 0.36. Patients with concentric left ventricular (LV) remodeling have a cardiovascular risk intermediate between those with normal left ventricular structure and those with left ventricular hypertrophy.

One outcome of treatment of diabetes mellitus while minimizing cardiac hypertrophy or left ventricular hypertrophy may be a decrease in ventricular mass. Another outcome of treatment of diabetes mellitus while minimizing cardiac hypertrophy or left ventricular hypertrophy may be a decrease in the rate of increase of ventricular mass. Another outcome of treatment of diabetes mellitus while minimizing cardiac hypertrophy or left ventricular hypertrophy may be a decrease in left ventricular wall thickness. Another outcome of treatment of diabetes mellitus while minimizing cardiac hypertrophy or left ventricular hypertrophy may be a decrease in the rate of increase in left ventricular wall thickness.

The term “obesity” as used herein is a condition in which there is an excess of body fat. The definition of obesity is based on the Body Mass Index (BMI), which is calculated as body weight per height in meters squared (kg/m²). In Europe and the U.S.A., “obesity” means a condition whereby a healthy subject has a Body Mass Index (BMI) greater than or equal to 30 kg/m², or a condition whereby a subject with at least one complication has a BMI greater than or equal to 27 kg/m². A “subject at risk of obesity” means a healthy subject with a BMI of 25 kg/m² or more but less than 30 kg/m² or a subject with at least one complication with a BMI of 25 kg/m² or more but less than 27 kg/m².

The risks associated with obesity occur at a low Body Mass Index (BMI) in Asians, compared to that in Westerners. In Asian countries, including Japan, “obesity” means a condition whereby a subject with at least one obesity-induced or obesity-related complication, that requires weight reduction or that would be improved by weight reduction, has a BMI greater than or equal to 25 kg/m². In Asian countries, a “subject at risk of obesity” is a subject with a BMI of 23 kg/m² or more but less than 25 kg/m².

As used herein, the term “obesity” is meant to encompass all of the above definitions of obesity.

Obesity-induced or obesity-related complications include, but are not limited to, diabetes mellitus, abnormal glucose tolerance, insulin-resistance syndrome, dyslipidemia, hypertension, hyperuricemia, gout, coronary artery disease, myocardial infarction, angina pectoris, sleep apnea syndrome, Pickwickian syndrome, fatty liver, cerebral infarction, cerebral thrombosis, transient ischemic attack, orthopedic disorders, degenerative arthritis, lumbago, emmeniopathy and infertility. In particular, complications include: hypertension, hyperlipidemia, dyslipidemia, abnormal glucose tolerance, cardiovascular diseases, sleep apnea syndrome, diabetes mellitus and other obesity-related conditions.

Treatment of obesity and obesity-related disorders means the administration of the compounds or mixture compositions of the present invention to reduce or maintain the body weight of an obese patient. One outcome of treatment may be reducing the body weight of an obese patient relative to that subject's body weight immediately before the administration of the compounds or mixture compositions according to the present invention. Another outcome of treatment may be maintaining body weight previously lost as a result of diet, exercise, or pharmacotherapy. Another outcome of treatment may be decreasing the occurrence of and/or the severity of obesity-related diseases. The treatment may result in a reduction in food and/or calorie intake, including a reduction in total food intake, or a reduction of intake of specific components of the diet such as carbohydrates or fats; and/or the inhibition of nutrient absorption; and/or the inhibition of the reduction of metabolic rate. The treatment may also result in an alteration of metabolic rate, such as an inhibition of the reduction of metabolic rate or an increase in metabolic rate; and/or in minimization of the metabolic resistance that typically results from weight loss.

Prevention of obesity and obesity-related disorders means the administration of the compounds or mixture compositions of the present invention to reduce or maintain the body weight of a subject at risk of obesity. One outcome of prevention may be reducing the body weight of a subject at risk of obesity relative to that subject's body weight immediately before the administration of the compounds or mixture compositions according to the present invention. Another outcome of prevention may be maintaining body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, type 2 diabetes mellitus, polycystic ovary syndrome, cardiovascular diseases, degenerative arthritis, dermatological disorders, hypertension, insulin resistance, hypercholesterolemia, hypertriglyceridemia and gallstone disease.

The term “atherosclerosis” as used herein encompasses vascular diseases and conditions that are recognized and understood by physicians practicing in the relevant fields of medicine. Atherosclerosis, coronary heart disease (also known as coronary artery disease or ischemic heart disease), cerebrovascular disease and peripheral vasodilatation diseases are all clinical manifestations of atherosclerosis and are therefore encompassed by the terms “atherosclerosis” and “atherosclerotic disease.” The composition of a therapeutically effective amount of an anti-obesity agent in combination with a therapeutically effective amount of an anti-diabetic agent may be administered to prevent or reduce the risk of occurrence or recurrence, where the potential exists, of coronary heart disease, cerebrovascular disease or intermittent claudication. Coronary heart disease events are intended to include CHD death, myocardial infarction (such as heart attack) and revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events and intermittent claudication. It is intended that persons who have previously experienced one or more non-fatal atherosclerotic disease events are those for whom the potential for recurrence of such an event exists.

Circadian rhythms affect physiological parameters. Physiological parameters include rest-activity, sleep-wake cycles, body temperature, rhythms in hormone levels, and oscillations in general physiology. When these parameters are out of synchrony with the daily clock, a circadian rhythm imbalance occurs which can affect physiology, performance on a variety of tasks and one's emotional well being. The present invention is useful, for example, in the prevention or treatment of conditions associated with circadian rhythmicity as well as mental and physical disorders associated with travel across time zones and with rotating shift-work schedules.

In another embodiment, the present invention provides a method for the prevention or treatment of a circadian rhythm disorder in a mammal, including syndrome, shift-work sleep disorder, delayed sleep-phase syndrome, advanced sleep-phase syndrome, and non-24-hour sleep-wake disorder.

In another embodiment, the present invention provides a method for shortening the time of re-entrainment (return to normal entrainment of the circadian rhythms; synchronized to the environmental light-dark cycle) in a subject following an irregular shift in the sleep-wake cycle.

In another embodiment, the present invention provides a method for alleviating the effects of jet lag in a traveler. The purpose of this embodiment is to assist the body to adjust physiologically to the changes in sleep and eating patterns when crossing several time zones.

In a preferred embodiment, the present invention provides a method for resetting the internal circadian clock in a patient to match the patient's current activity/sleep cycle. For example, such a method is effective for shift workers changing from a day to a night shift or vice versa.

The present invention provides a method for enhancing or improving sleep quality by increasing sleep efficiency and augmenting sleep maintenance. In addition, the present invention provides a method for preventing and treating sleep disorders and sleep disturbances. The present invention further provides a pharmaceutical composition for enhancing or improving sleep quality and increasing sleep efficiency and sleep maintenance. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during drug and alcohol withdrawal stages), childhood onset DIMS, nocturnal myoclonus and restless legs and non specific REM (eye movement) disturbances as seen in ageing.

The following outcomes in a patient which are provided by the present invention may be related to improvement in sleep quality: an increase in value which is calculated from the time that a subject sleeps divided by the time that a subject is attempting to sleep; a decrease in sleep latency (the time it takes to fall asleep); a decrease in the number of awakenings during sleep; a decrease in the time spent awake following the initial onset of sleep; an increase in the total amount of sleep; an increase the amount and percentage of REM sleep; an increase in the duration and occurrence of REM sleep; a reduction in the fragmentation of REM sleep; an increase in the amount and percentage of slow-wave (for example, stage 3 or 4) sleep; an increase in the amount and percentage of stage 2 sleep; a decrease in the number of awakenings, especially in the early morning; an increase in daytime alertness; and increased sleep maintenance. Secondary outcomes which may be provided by the present invention include enhanced cognitive function and increased memory retention. “Method for enhancing the quality of sleeps” means a method that results in outcomes in a patient which may be related to enhancement in sleep quality, including, but not limited to, the outcomes correlated to enhancement of sleep quality as defined above.

The present invention is further useful for the prevention and treatment of sleep disorders and sleep disturbances including sleep problems associated with insomnia, hypersomnia, sleep apnea syndrome, narcolepsy, nocturnal myoclonus, REM sleep interruptions, jet-lag, shift workers' sleep disturbances, dysomnias, noctiphobia, night eating syndrome, insomnias associated with depression or with emotional/mood disorders, dysfunctions associated with sleep (parasomnias), as well as sleep walking and enuresis, as well as sleep disorders which accompany aging. Sleep disorders and sleep disturbances are generally characterized by difficulty in initiating or maintaining sleep or in obtaining restful or enough sleep.

In addition, certain drugs may also cause reductions in REM sleep as a side effect and the present invention may be used to correct those types of sleeping disorders as well. The present invention would also be of benefit in the treatment of syndromes such as fibromyalgia which are manifested by non-restorative sleep and muscle pain or sleep apnea which is associated with respiratory disturbances during sleep. It will be clear that the present invention is not limited to just sleep disorders and sleep disturbances, but is applicable to a wide variety of conditions which result from a diminished quality of sleep.

Compounds of the present invention and compositions thereof, or combinations of these and other drugs, are useful for treating and preventing these conditions.

In the present invention, a subject mammal is preferably a human. Although the present invention is applicable for both old and young people, it may find greater application in elderly people. Further, although the invention may be employed to enhance the sleep of healthy people, it may be especially beneficial for enhancing the sleep quality of people suffering from sleep disorders or sleep disturbances.

In the compounds of the formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of the formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within the formula I, can be prepared without undue experimentation by conventional techniques well known to those skilled in the art or by processes analogous to those described in the Schemes and Examples herein using appropriate isotopically-enriched reagents and/or intermediates.

The compositions according to the present invention may be used in combination with other drugs that may also be useful in the treatment, prevention or control of disorders, such as hypertension, hypertension associated with obesity, hypertension-related disorders, cardiac hypertrophy, left ventricular hypertrophy, and metabolic syndrome, obesity and obesity-related disorders. Such other drugs may be administered, by a route and in an amount commonly used therefore, concurrently or sequentially with a composition according to the present invention. When a composition of the present invention is used concurrently with one or more other drugs, a pharmaceutical composition in unit dosage form containing such other drugs and the composition of the present invention is preferred.

However, the combination therapy also includes therapies in which the composition according to the present invention and one or more other drugs are administered on different overlapping dosage schedules. It is also contemplated that when used in combination with one or more other active ingredients, the composition according to the present invention and the other active ingredients may be used in lower doses than when each is used singly. Accordingly, the pharmaceutical compositions of the present invention include those that contain one or more other active ingredients, in addition to a composition of the present invention.

Examples of other active ingredients that may be administered in combination with a composition according to the present invention, and either administered separately or in the same pharmaceutical composition, include, but are not limited to:

(a) anti-diabetic agents such as (i) PPARγ agonists such as glitazones (for example ciglitazone, darglitazone, englitazone, isaglitazone (MCC-555), pioglitazone, rosiglitazone, troglitazone, BRL49653, CLX-0921 and 5-BTZD), GW-0207, LG-100641 and LY-300512; (ii) biguanides such as buformin, metformin and phenformin; (iii) protein tyrosine phosphatase-1B (PTP-1B) inhibitors; (iv) sulfonylureas such as acetohexamide, chlorpropamide, diabinese, glybenclamide, glipizide, glyburide, glimepiride, gliclazide, glipentide, gliquidone, glisolamide, tolazamide and tolbutamide; (v) meglitinides such as repaglinide and nateglinide; (vi) alpha glucoside hydrolase inhibitors such as acarbose, adiposine, camiglibose, emiglitate, miglitol, voglibose, pradimicin-Q, salbostatin, CKD-711, MDL-25,637, MDL-73,945 and MOR 14; (vii) alpha-amylase inhibitors such as tendamistat, trestatin and Al-3688; (viii) insulin secreatagogues such as linogliride and A-4166; (ix) fatty acid oxidation inhibitors, such as clomoxir and etomoxir; (x) A2 antagonists, such as midaglizole, isaglidole, deriglidole, idazoxan, earoxan and fluparoxan; (xi) insulin or insulin mimetics, such as biota, LP-100, novarapid, insulin detemir, insulin lispro, insulin glargine, insulin zinc suspension (lente and ultralente), Lys-Pro insulin, GLP-1 (73-7) (insulin tropin) and GLP-1 (7-36)-NH₂; (xii) non-thiazolidinediones such as JT-501 and farglitazar (GW-2570/GI-262579); (xiii) PPARα/γ dual agonists such as MK-0767, CLX-0940, GW-1536, GW-1929, GW-2433, KRP-297, L-796449, LR-90 and SB219994; (xiv) other insulin sensitizing drugs; and (xv) VPAC2 receptor agonists;

(b) lipid lowering agents such as (i) bile acid sequestrants such as cholestyramine, colesevelem, colestipol, dialkylaminoalkyl derivatives of a cross-linked dextran, Colestid®, LoCholest® and Questran®; (ii) HMG-CoA reductase inhibitors such as atorvastatin, itavastatin, fluvastatin, lovastatin, pravastatin, rivastatin, rosuvastatin, simvastatin and ZD-4522; (iii) HMG-CoA synthase inhibitors; (iv) cholesterol absorption inhibitors such as stanol esters, beta-sitosterol, sterol glycosides such as tiqueside, and azetidinones such as ezetimibe; (v) acyl coenzyme A-cholesterol acyl transferase (ACAT) inhibitors such as avasimibe, eflucimibe, KY505 and SMP797; (vi) CETP inhibitors such as H7705, torcetrapib, CP532,632, BAY63-2149, SC591 and SC795; (vii) squalene synthetase inhibitors; (viii) anti-oxidants such as probucol; (ix) PPARα agonists, such as beclofibrate, benzafibrate, ciprofibrate, clofibrate, etofibrate, fenofibrate, gemcabene, gemfibrozil, GW7647, BM170744, LY518674, and other fibric acid derivatives such as Atromid®, Lopid® and Tricor®; (x) FXR receptor modulators such as GW4064 and SR103912; (xi) LXR receptor such as GW3965, T9013137 and XTC0179628; (xii) lipoprotein synthesis inhibitors such as niacin; (xiii) rennin-angiotensin system inhibitors; (xiv) PPARδ partial agonists; (xv) bile acid reabsorption inhibitors, such as BARI1453, SC435, PHA384640, S8921 and AZD7706; (xvi) PPARδ agonists such as GW501516 and GW590735; (xvii) triglyceride synthesis inhibitors; (xviii) microsomal triglyceride transport (MTTP) inhibitors, such as inplitapide, LAB687 and CP346086; (xix) transcription modulators; (xx) squalene epoxidase inhibitors; (xxi) low density lipoprotein (LDL) receptor inducers; (xxii) antiplatelet drugs; (xxiii) 5-LO or FLAP inhibitors; and (xxiv) niacin receptor agonists;

(c) anti-hypertensive agents such as (i) diuretics, such as thiazides, including chlorthalidone, chlorthiazide, dichlorophenamide, hydroflumethiazide, indapamide and hydrochlorothiazide; loop diuretics, such as bumetanide, ethacrynic acid, furosemide and torsemide; potassium sparing agents, such as amiloride and triamterene; and aldosterone antagonists, such as spironolactone and epirenone; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol and timolol; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine and verapamil; (iv) angiotensin converting enzyme (ACE) inhibitors such as benazepril, captopril, cilazapril, delapril, enalapril, fosinopril, imidapril, losinopril, moexipril, quinapril, quinaprilat, ramipril, perindopril, perindropril, quanipril, spirapril, tenocapril, trandolapril and zofenopril; (v) neutral endopeptidase inhibitors such as omapatrilat, cadoxatril, ecadotril, fosidotril, sampatrilat, AVE7688 and ER4030; (vi) endothelin antagonists such as tezosentan, A308165 and YM62899; (vii) vasodilators such as hydralazine, clonidine, minoxidil and nicotinyl alcohol; (viii) angiotensin II receptor antagonists such as candesartan, eprosartan, irbesartan, losartan, pratosartan, tasosartan, telmisartan, valsartan, EXP-3137, FI6828K and RNH6270; (ix) α/β adrenergic blockers as nipradilol, arotinolol and amosulalol; (x) alpha 1 blockers, such as terazosin, urapidil, prazosin, bunazosin, trimazosin, doxazosin, naftopidil, indoramin, WHIP164 and XEN010; (xi) alpha 2 agonists such as lofexidine, tiamenidine, moxonidine, rilmenidine and guanobenz, and the like; and (xii) aldosterone inhibitors;

(d) anti-obesity agents, such as (i) 5HT (serotonin) transporter inhibitors, such as paroxetine, fluoxetine, fenfluramine, fluvoxamine, sertraline and imipramine; (ii) NE (norepinephrine) transporter inhibitors, such as GW320659, despiramine, talsupram and nomifensine; (iii) CB-1 (cannabinoind-1 receptor) antagonist/inverse agonists, such as rimonabant (Sanofi Synthelabo), SR-147778 (Sanofi Synthelabo), BAY65-2520 (Bayer) and SLV319 (Solvay), and those disclosed in U.S. Pat. Nos. 5,532,237, 4,973,587, 5,013,837, 5,081,122, 5,112,820, 5,292,736, 5,624,941 and 6,028,084; and WO96/33159, WO98/33765, WO98/43636, WO98/43635, WO01/09120, WO01/96330, WO98/31227, WO98/41519, WO98/37061, WO00/10967, WO00/10968, WO97/29079, WO99/02499, WO01/58869, WO02/076949, WO01/64632, WO01/64633, WO03/006007 and WO03/007887; and EPO Application No. EP-658546; (iv) ghrelin antagonists, such as those disclosed in WO01/87335 and WO02/08250; (v) H3 (histamine H3) antagonist/inverse agonists, such as thioperamide, 3-(1H-imidazol-4-yl]propyl N-(4-pentenyl)carbamate, clobenpropit, iodophenpropit, imoproxifan, GT2394 (Gliatech) and A331440, and those disclosed in WO02/15905; and O-[3-(1H-imidazol-4-yl)propanol]carbamates [Kiec-Kononowicz, K. et al., Pharmazie, 55:349-55 (2000)], piperidine-containing histamine H3-receptor antagonists [Lazewska, D. et al., Pharmazie, 56:927-32 (2001)], benzophenone derivatives and related compounds [Sasse, A. et al., Arch. Pharm. (Weinheim) 334:45-52 (2001)], substituted N-phenylcarbamates (Reidemeister, S. et al., Pharmazie, 55:83-6 (2000)], and proxifan derivatives [Sasse, A. et al., J. Med. Chem. 43:3335-43 (2000)]; (vi) melanin-concentrating hormone 1 receptor (MCH1R) antagonists, such as T-226296 (Takeda), SNP-7941 (Synaptic), and those disclosed in WO01/82924, WO01/87834, WO02/051809, WO02/06245, WO02/076929, WO02/076947, WO02/04433, WO02/51809, WO02/083134, WO02/094799, WO03/004027, and Japanese Patent Application No. JP13226269; (vii) MCH2R (melanin concentrating hormone 2R) agonist/antagonists; (viii) NPY1 (neuropeptide Y Y1) antagonists, such as BIBP3226, 2-[1-(5-chloro-3-isopropyloxycarbonylaminophenyl)ethylamino]-6-[2-(5-ethyl-4-methyl-1,3-thiazol-2-yl)ethyl]-4-morpholinopyridine, BIB03304, LY-357897, CP-671906 and GI-264879A; and those disclosed in U.S. Pat. No. 6,001,836; and WO96/14307, WO01/23387, WO99/51600, WO01/85690, WO01/85098, WO01/85173 and WO01/89528; (ix) NPY5 (neuropeptide Y Y5) antagonists, such as L-152,804, GW-569180A, GW-594884A, GW-587081X, GW-548118X, FR 235,208, FR-226928, FR240662, FR252384, 1229U91, GI-264879A, CGP71683A, LY-377897, LY366377, PD-160170, SR-120562A, SR-120819A, JCF-104 and H409/22; and those compounds disclosed in U.S. Pat. Nos. 6,140,354, 6,191,160, 6,258,837, 6,313,298, 6,337,332, 6,329,395 and 6,340,683; U.S. Pat. Nos. 6,326,375, 6,329,395, 6,337,332 and 6,335,345; European Patent Nos. EP-01010691 and EP-01044970; and PCT International Patent Publication Nos. WO97/19682, WO97/20820, WO97/20821, WO97/20822, WO97/20823, WO98/27063, WO00/107409, WO00/185714, WO00/185730, WO00/64880, WO00/68197, WO00/69849, WO01/09120, WO01/14376, WO01/85714, WO01/85730, WO01/07409, WO01/02379, WO01/23388, WO01/23389, WO01/44201, WO01/62737, WO01/62738, WO01/09120, WO02/20488, WO02/22592, WO02/48152, WO02/49648 and WO02/094789; and Norman et al., J. Med. Chem. 43:4288-4312 (2000); (x) leptin, such as recombinant human leptin (PEG-OB, Hoffman La Roche) and recombinant methionyl human leptin (Amgen); (xi) leptin derivatives, such as those disclosed in U.S. Pat. Nos. 5,552,524; 5,552,523; 5,552,522; and 5,521,283; and PCT International Publication Nos. WO96/23513, WO96/23514, WO96/23515, WO96/23516, WO96/23517, WO96/23518, WO96/23519 and WO96/23520; (xii) opioid antagonists, such as nalmefene (Revex®), 3-methoxynaltrexone, naloxone and naltrexone; and those disclosed in WO00/21509; (xiii) orexin antagonists, such as SB-334867-A; and those disclosed in WO01/96302, WO01/68609, WO02/51232, WO02/51838 and WO03/023561; (xiv) BRS3 (bombesin receptor subtype 3) agonists; (xv) CCK-A (cholecystokinin-A) agonists, such as AR-R15849, GI181771, JMV-180, A-71378, A-71623 and SR146131, and those disclosed in U.S. Pat. No. 5,739,106; (xvi) CNTF (ciliary neurotrophic factors), such as GI-181771 (Glaxo-SmithKline); SR146131 (Sanofi Synthelabo); butabindide; and PD170292 and PD149164 (Pfizer); (xvii) CNTF derivatives, such as axokine (Regeneron); and WO94/09134, WO98/22128 and WO99/43813; (xviii) GHS (growth hormone secretagogue receptor) agonists, such as NN703, hexarelin, MK-0677, SM-130686, CP-424,391, L-692,429 and L-163,255, and those disclosed in U.S. Pat. No. 6,358,951, U.S. Patent Application Nos. 2002/049196 and 2002/022637; and WO01/56592, and WO02/32888; (xix) 5HT2c (serotonin receptor 2c) agonists, such as BVT933, DPCA37215, IK264, PNU22394, WAY161503, R-1065 and YM 348; and those disclosed in U.S. Pat. No. 3,914,250; and WO02/36596, WO02/48124, WO02/10169, WO01/66548, WO02/44152, WO02/51844, WO02/40456 and WO02/40457; (xx) Mc3r (melanocortin 3 receptor) agonists; (xxi) Mc4r (melanocortin 4 receptor) agonists, such as CHIR86036 (Chiron); ME-10142 and ME-10145 (Melacure), and those disclosed in WO99/64002, WO00/74679, WO01/991752, WO01/74844, WO01/70708, WO01/70337, WO01/91752, WO02/059095, WO02/059107, WO02/059108, WO02/059117, WO02/12166, WO02/11715, WO02/12178, WO02/15909, WO02/068387, WO02/068388, WO02/067869, WO03/007949 and WO03/009847; (xxii) monoamine reuptake inhibitors, such as sibutratmine (Meridia®/Reductil®) and a salt thereof, and those compounds disclosed in U.S. Pat. Nos. 4,746,680, 4,806,570 and 5,436,272, and U.S. Patent Publication No. 2002/0006964, and WO01/27068 and WO01/62341; (xxiii) serotonin reuptake inhibitors, such as dexfenfluramine, fluoxetine, and those in U.S. Pat. No. 6,365,633, and WO01/27060 and WO01/162341; (xxiv) GLP-1 (glucagon-like peptide 1) agonists; (xxv) topiramate (Topimax®); (xxvi) phytopharm compound 57 (CP 644,673); (xxvii) ACC2 (acetyl-CoA carboxylase-2) inhibitors; (xxviii) β3 (beta adrenergic receptor 3) agonists, such as AD9677/TAK677 (Dainippon/Takeda), CL-316,243, SB418790, BRL-37344, L-796568, BMS-196085, BRL-35135A, CGP12177A, BTA-243, GW427353, trecadrine, zeneca D7114 and SR59119A, and those disclosed in U.S. Pat. Application Nos. 5,705,515, U.S. Pat. No. 5,451,677, and WO01/74782 and WO02/32897; (xxix) DGAT1 (diacylglycerol acyltransferase 1) inhibitors; (N. xx) DGAT2 (diacylglycerol acyltransferase 2) inhibitors; (xxxi) FAS (fatty acid synthase) inhibitors, such as cerulenin and C75; (xxxii) PDE (phosphodiesterase) inhibitors, such as theophylline, pentoxifylline, zaprinast, sildenafil, aminone, milrinone, cilostamide, rolipram and cilomilast; (xxxiii) thyroid hormone β agonists, such as KB-2611 (KaroBioBMS), and those disclosed in WO02/15845; and Japanese Patent Application No. JP2000256190; (xxxiv) UCP-1 (uncoupling protein 1), 2 or 3 activators, such as phytanic acid, 4-[(E)-2-(5,6,7,8-tetrahydro-5,5,8,8-tetramethyl-2-napthalenyl)-1-propenyl]benzoic acid (TTNPB) and retinoic acid; and those disclosed in WO99/00123; (xxxv) acyl-estrogens, such as oleoyl-estrone, disclosed in del Mar-Grasa, Metal., Obesity Research, 9:202-9 (2001); (xxxvi) glucocorticoid antagonists; (xxxvii) 11βHSD-1 (11-beta hydroxy steroid dehydrogenase type 1) inhibitors, such as BVT3498, BVT2733, and those compounds disclosed in WO01/90091, WO01/90090 and WO01/90092; (xxxviii) SCD-1 (stearoyl-CoA desaturase-1) inhibitors; (xxxix) dipeptidyl peptidase IV (DP-IV) inhibitors, such as isoleucine thiazolidide, valine pyrrolidide, NVP-DPP728, LAF237, P93/01, TSL225, TMC-2A/2B/2C, FE999011, P9310/K364, VIP0177, SDZ274-444; and the compounds disclosed in WO03/004498, WO03/004496, EP1258476, WO02/083128, WO02/062764, WO03/000250, WO03/002530, WO03/002531, WO03/002553, WO03/002593, WO03/000180 and WO03/000181; (xxxx) lipase inhibitors, such as tetrahydrolipstatin (Orlistat/Xenical®), Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC80267, and those disclosed in WO01/77094, and U.S. Pat. Nos. 4,598,089, 4,452,813, 5,512,565, 5,391,571, 5,602,151, 4,405,644, 4,189,438 and 4,242,453; (mood) fatty acid transporter inhibitors; (xxxxii) dicarboxylate transporter inhibitors; (xxxxiii) glucose transporter inhibitors; (xxxxiv) phosphate transporter inhibitors; (xxxxv) melanocortin agonists, such as melanotan II or those described in WO99/64002 and WO00/746799; (xxxxvi) melanin concentrating hormone antagonists; (xxxxvii) galanin antagonists; (xxxxviii) CCK agonists; (xxxxix) corticotropin-releasing hormone agonists; and (xxxxx) phosphodiesterase-3B (PDE3B) inhibitors.

The above combinations include combinations of a composition of the present invention not only with one other active compound, but also with two or more other active compounds. There are many examples including combinations of the compositions of the present invention with one, two or more active compounds selected from lipid-lowering agents and anti-hypertensive agents. Combinations of the compositions of the present invention with one, two or more active compounds selected from lipid lowering agents and anti-diabetic agents are useful to treat, control or prevent metabolic syndrome. In particular, compositions including an anti-obesity agent, an anti-hypertensive agent, in addition to an anti-diabetic agent and/or a lipid lowering agent will be useful to synergistically treat, control or prevent metabolic syndrome.

### EXAMPLES

The present invention is further specifically described below referring to Formulation Examples, Examples and Reference Examples, but is not limited thereto.

### Formulation Example 1

The compound (20.0 g) of Example 1, lactose (417 g), crystalline cellulose (80 g) and partially pregelatinized starch (80 g) are mixed using a V-blender. To the mixture is then added magnesium stearate (3.0 g) and the whole is mixed. The mixed powder is tableted in accordance with a conventional method to obtain 3,000 tablets having a diameter of 7.0 mm and a weight of 150 mg per tablet.

### The Content of One Tablet (150 mg)

the compound of Example 15.0 mg
lactose 104.25 mg
crystalline cellulose 20.0 mg
partially pregelatinized starch 20.0 mg
magnesium stearate 0.75 mg

### Formulation Example 2

In 172.5 grams of purified water are dissolved 10.8 grams of hydroxypropylcellulose 2910 and 2.1 grams of polyethylene glycol 6000. To the solution is dispersed 2.1 grams of titanium dioxide to prepare a coating liquid. Using HICOATER-MINI, 2,500 tablets prepared in Formulation Example 1 are subjected to spray-coating with the coating liquid to provide a film coated tablet with a weight of 155 mg.

### The Content of One Tablet 0155 mg)

the tablet prepared in Formulation Example 1150 mg
hydroxypropylcellulose 2910 3.6 mg
polyethylene glycol 6000 0.7 mg
titanium dioxide 0.7 mg

In Reference Examples and Examples, thin-layer chromatography employed Silica Gel 60 F₂₅₄ (Merck) as a plate, whereas thin-layer chromatography based on amine employed PLC05 NH (FUJI Silysia) as a plate and a UV detector for a detection method. Wako Gel™ C-300 (Wako Pure Chemical Industries) was used for silica gel for column; and a cartridge for FLASH, KP-SIL or KP-NH (Biotage Japan) or Purif-pack SI or Purif-pack NH (Moritex), was used for a charged silica gel column. In addition, NMR spectra were measured using FT NMR “JNM-AL-400” (JEOL); and mass spectra were measured using Quattro II (Micromass).

The meanings of the abbreviations in Examples described below are shown below. i-Bu: isobutyl; n-Bu: n-butyl; t-Bu: tert-butyl; Me: methyl; Et: ethyl; Ph: phenyl; i-Pr: isopropyl; n-Pr: n-propyl; CDCl₃: heavy chloroform; CD₃OD: heavy methanol; DMSO-d₆: heavy dimethylsulfoxide.

The meanings of the abbreviations in the nuclear magnetic resonance spectra are shown below.

s: singlet; d: doublet; dd: double doublet; dt: double triplet; t: triplet; m: multiplet; br: broad; brs: broad singlet; q: quartet; J: coupling constant; and Hz: hertz.

### Reference Example 1-1

### 8-azabicyclo[3,2,1]octan-3-one hydrochloride

In 1,2-dichloroethane (100 mL) was dissolved tropinone (5.00 g). To the solution was added 1-chloroethylchloroformate (4.70 mL) at 0° C., followed by stirring the mixture under heating to reflux for 6 hours. Methanol (100 mL) was added to the residue at room temperature, and the mixture was stirred under heating to reflux for 2 hours. The reaction solution was concentrated under reduced pressure, and the residue was then washed with diethyl ether to give the title compound (4.57 g) as a pale yellow solid. mass: 126 (M+1)⁺.

### Reference Example 1-2

### 3-oxo-8-azabicyclo[3,2,1]octan-8-carboxylic acid benzyl ester

In chloroform (70.0 mL) was suspended 8-azabicyclo[3,2,1]octan-3-one hydrochloride (4.57 g) obtained in Reference Example 1-1. To the suspension were added triethylamine (15.7 mL) and benzyloxycarbonyl chloride (56.6 mL) at 0° C., and the mixture was then stirred overnight at room temperature. A saturated aqueous sodium hydrogencarbonate solution was added to the reaction solution, the mixture was extracted with chloroform, and the organic layer was dried over anhydrous sodium sulfate. Insoluble matters were filtered out, and the filtrate was concentrated under reduced pressure. The resultant residue was purified by silica gel column chromatography (hexane/ethyl acetate=75:25 to 50:50) to yield the title compound (2.77 g) as a pale yellow oil. mass: 260 (M+1)⁺.

### Reference Example 1-3

### 3,3-difluoro-8-azabicyclo[3,2,1]octan-8-carboxylic acid benzyl ester

In toluene (30.0 mL) was dissolved 3-oxo-8-azabicyclo[3,2,1]octan-8-carboxylic acid benzyl ester (2.51 g) obtained in Reference Example 1-2. To the solution was added diethylaminosulfur trifluoride (6.39 mL) under nitrogen atmosphere at 0° C., and the mixture was stirred at 100° C. for 24 hours. A saturated aqueous sodium hydrogencarbonate solution was added to the reaction solution, and insoluble matters were filtered out. The filtrate was extracted with ethyl acetate, and the organic layer was washed with a saturated saline solution and then dried over anhydrous sodium sulfate. Insoluble matters were filtered out, and the filtrate was concentrated under reduced pressure. The resultant residue was purified by silica gel column chromatography (hexane/ethyl acetate=90:10 to 50:50) to yield the title compound (0.777 g) as a pale yellow oil. mass: 282 (M+1)⁺.

### Reference Example 1-4

### 3,3-difluoro-8-azabicyclo[3,2,1]octane hydrochloride

In methanol (10.0 mL) was dissolved 3,3-difluoro-8-azabicyclo[3,2,1]octan-8-carboxylic acid benzyl ester (777 mg) obtained in Reference Example 1-3. To the solution were added 20 cyclohexene (2.80 mL) and 10% palladium carbon (300 mg) at room temperature, and the mixture was stirred under heating to reflux for 1 hour. Insoluble matters were filtered out, a 10% hydrochloric acid-methanol solution (10.0 mL) was added to the filtrate, and the mixture was then concentrated under reduced pressure to give the title compound (409 mg) as a pale yellow solid. mass: 148 (M+1)⁺.

### Reference Example 1-5

### 3,3′-dithiobis(5-methyl-1H-pyrazole)

In tetrahydrofuran (500 mL) was suspended tert-butoxy potassium (22.9 g). To the suspension was added dropwise acetone (5.00 mL) at room temperature. The mixture was stirred for 10 minutes, followed by slowly dropwise adding a solution of carbon disulfide (4.11 mL) in tetrahydrofuran (50.0 mL) at room temperature. The mixture was stirred for 2 hours. A 4N hydrochloric acid-dioxane solution (51.0 mL) was added to the reaction solution at 0° C., and the mixture was stirred at the same temperature for 30 minutes. Insoluble matters were filtered out, and the filtrate was concentrated under reduced pressure to give a crude product of 3-oxybutane dithionic acid. In ethanol (150 mL) was dissolved the resultant 3-oxybutane dithionic acid. To the solution was added dropwise hydrazine monohydrate (3.41 g) at 0° C. The mixture was stirred under heating to reflux for 6 hours and further stirred at room temperature for 12 hours. The reaction solution was concentrated under reduced pressure, a saturated aqueous sodium hydrogencarbonate solution was added to the resultant residue, and the mixture was extracted with ethyl acetate. The organic layer was washed with a saturated saline solution and then dried over anhydrous sodium sulfate. Insoluble matters were filtered out, the filtrate was concentrated under reduced pressure, and the resultant residue was purified by silica gel column chromatography (hexane/ethyl acetate=2:1 to 0:1) to yield the title compound (2.03 g) as a dark brown solid. mass: 227 (M+1)⁺.

### Reference Example 1-6

### 3,3′-dithiobis(1,5-dimethyl-1H-pyrazole)

In dimethylformamide (25.0 mL) was dissolved 3,3′-dithiobis(5-methyl-1′-1-pyrazole) (1.50 g) obtained in Reference Example 1-5. To the solution were added methyl iodide (1.04 mL) and cesium carbonate (6.48 g), and the mixture was stirred overnight at room temperature. Water was added to the reaction solution, the mixture was extracted with ethyl acetate, and the organic layer was washed with water and a saturated saline solution and then dried over anhydrous sodium sulfate. Insoluble matters were filtered out, the filtrate was concentrated under reduced pressure, and the resultant residue was purified by silica gel column chromatography (hexane/ethyl acetate=2:1 to 0:1) to yield the title compound (492 mg) as a dark brown solid. mass: 255 (M+1)⁺.

### Reference Example 1-7

### 1,5-dimethyl-1,2-dihydro-3H-pyrazol-3-thione

In tetrahydrofuran (10.0 mL) was dissolved 3,3′-dithiobis(1,5-dimethyl-1H-pyrazole) (492 mg) obtained in Reference Example 1-6. To the solution was added a 1M aqueous sodium hydrosulfite solution (19.3 mL), and the mixture was stirred at room temperature for 6 hours. The reaction solution was extracted with chloroform and washed with a saturated saline solution, followed by being dried over anhydrous sodium sulfate. Insoluble matters were filtered out, and the filtrate was concentrated under reduced pressure to give a crude product (240 mg) of the title compound as a pale yellow solid. mass: 129 (M+1)⁺.

### Reference Example 1-8

### 2-tert-butyl-5-ethyl-2,4-dihydro-3H-pyrazol-3-one

In acetic acid (50.0 mL) was dissolved tert-butyl hydrazine hydrochloride (4.99 g). To the solution was added 3-methyl oxopentanoate ester (5.03 mL) at room temperature, and the mixture was then stirred overnight at 110° C. The reaction solution was concentrated under reduced pressure, and a saturated aqueous sodium hydrogencarbonate solution was then added to the residue. The mixture was extracted with ethyl acetate, and the organic layer was washed with a saturated saline solution and then dried over anhydrous sodium sulfate. Insoluble matters were filtered out, and the filtrate was concentrated under reduced pressure. The residue was washed with diethyl ether/hexane (1:1) to give the title compound (4.32 g) as a white solid. mass: 169 (M+1)⁺.

### Reference Example 1-9

### 5-ethyl-1,2-dihydro-3H-pyrazol-3-thione hydrochloride

In toluene (15.0 mL) was dissolved 2-tert-butyl-5-ethyl-2,4-dihydro-3H-pyrazol-3-one (1.00 g) obtained in Reference Example 1-8. To the solution was added Lawesson's reagent (1.44 g) at room temperature, and the mixture was then stirred at 100° C. for 4 hours. After concentration under reduced pressure of the reaction solution, a 2N aqueous sodium hydroxide solution (5.00 mL) was added to the residue, and the water layer was washed with diethyl ether. Concentrated hydrochloric acid (6.00 mL) was added to the water layer at room temperature, and the mixture was stirred overnight at 100° C. After concentration under reduced pressure of the reaction solution, toluene was added to the residue, and the mixture was concentrated under reduced pressure again. This operation was repeated three times to give the title compound (0.457 g) as a white solid. mass: 129 (M+1)⁺.

### Reference Example 1-10

### 2-methyl-3-oxopentanoic acid methyl ester

In tetrahydrofuran (70.0 mL) was dissolved 3-methyl oxopentanoate ester (5.05 mL). To the solution was added potassium carbonate (16.6 g) at room temperature, and the mixture was stirred under heating to reflux for 3 hours. Methyl iodide (3.00 mL) was added to the reaction solution at 0° C., and the mixture was stirred at room temperature for 8 hours. Insoluble matters were filtered out, and the filtrate was then concentrated under reduced pressure to give the title compound (5.38 g) as a pale yellow oil. mass: 145 (M+1)⁺.

### Reference Example 1-11

### 2-tert-butyl-5-ethyl-4-methyl-2,4-dihydro-3H-pyrazol-3-one

The title compound was obtained as a white solid by the same method as in Reference Example 1-8, methods equivalent thereto or combinations of these with usual methods using 2-methyl-3-oxopentanoic acid methyl ester obtained in Reference Example 1-10 instead of 3-oxopentanoic acid methyl ester. mass: 183 (M+1)⁺.

### Reference Example 1-12

### 5-ethyl-4-methyl-1,2-dihydro-3H-pyrazol-3-thione hydrochloride

The title compound was obtained as a white solid by the same method as in Reference Example 1-9, methods equivalent thereto or combinations of these with usual methods using 2-tert-butyl-5-ethyl-4-methyl-2,4-dihydro-3H-pyrazol-3-one obtained in Reference Example 1-11 instead of 2-tert-butyl-5-ethyl-2,4-dihydro-3H-pyrazol-3-one obtained in Reference Example 1-8. mass: 143 (M+1)⁺.

### Reference Example 1-13

### 2-oxobutanoic acid ethyl ester

In ethanol (200 mL) was dissolved 2-oxobutanoic acid (10.0 g). To the solution was added concentrated sulfuric acid (0.500 mL) at room temperature, and the mixture was then stirred overnight under heating to reflux. After concentration under reduced pressure of the reaction solution, a saturated aqueous sodium hydrogen-carbonate solution was added to the residue. The mixture was extracted with ethyl acetate, and the organic layer was washed with a saturated saline solution and then dried over anhydrous sodium sulfate. Insoluble matters were filtered out, and the filtrate was concentrated under reduced pressure to give the title compound (10.8 g) as a colorless oil. mass: 131 (M+1)⁺.

### Reference Example 1-14

### 3-bromo-2-oxobutanoic acid ethyl ester

Copper (II) bromide (46.3 g) was suspended in ethyl acetate (350 mL). To the suspension was added a solution of 2-oxobutanoic acid ethyl ester (10.8 g) obtained in Reference Example 1-13 in chloroform (100 mL) at room temperature, and the mixture was then stirred under heating to reflux for 18 hours. The reaction solution was filtered through silica gel, and the filtrate was concentrated under reduced pressure. The residue was purified by vacuum distillation (9 torr, 76-80° C.) to give the title compound (11.8 g) as a colorless oil. mass: 209 (M+1)⁺.

### Reference Example 1-15

### 5-methyl-2-thioxo-2,3-dihydro-1,3-thiazol-4-carboxylic acid ethyl ester

In ethanol (80.0 mL) was dissolved 3-bromo-2-oxobutanoic acid ethyl ester (5.00 g) obtained in Reference Example 1-14. To the solution was added ammonium carbamodithioate (2.64 g) at room temperature, and the mixture was stirred overnight under heating to reflux. After concentration under reduced pressure of the reaction solution, water was added to the residue. The precipitated solid was obtained through filtration and then washed with diethyl ether/hexane (1:1) to give the title compound (2.04 g) as a white solid. mass: 204 (M+1)⁺.

### Reference Example 1-16

### 2-oxopentanoic acid ethyl ester

The title compound was obtained as a colorless oil by the same method as in Reference Example 1-13, methods equivalent thereto or combinations of these with usual methods using 2-oxopentanoic acid instead of 2-oxobutanoic acid. mass: 145 (M+1)⁺.

### Reference Example 1-17

### 3-bromo-2-oxopentanoic acid ethyl ester

The title compound was obtained as a colorless oil by the same method as in Reference Example 1-14, methods equivalent thereto or combinations of these with usual methods using 2-oxopentanoic acid ethyl ester obtained in Reference Example 1-16 instead of 2-oxobutanoic acid ethyl ester obtained in Reference Example 1-13. mass: 223 (M+1)⁺.

### Reference Example 1-18

### 5-ethyl-2-thioxo-2,3-dihydro-1,3-thiazol-4-carboxylic acid ethyl ester

The title compound was obtained as a white solid by the same method as in Reference Example 1-15, methods equivalent thereto or combinations of these with usual methods using 3-bromo-2-oxopentanoic acid ethyl ester obtained in Reference Example 1-17 instead of 3-bromo-2-oxobutanoic acid ethyl ester obtained in Reference Example 1-14. mass: 218 (M+1)⁺.

### Reference Example 1-19

### 2-(cyclopropylmethyl)-1,3-dithiane-2-carboxylic acid ethyl ester

Sodium hydride (1.03 g) was suspended in toluene (40.0 mL), followed by adding a solution of ethyl 1,3-dithiane-2-carboxylate (4.13 g) and (bromomethyl)cyclopropane (2.52 mL) in dimethylformamide (10 mL) to the suspension at 0° C. and stirring the mixture overnight at room temperature. A saturated aqueous ammonium chloride solution was added to the reaction solution, and the mixture was extracted with ethyl acetate. The organic layer was washed with a saturated saline solution and then dried over anhydrous sodium sulfate. Insoluble matters were filtered out, and the filtrate was concentrated under reduced pressure to give the title compound (5.20 g) as a colorless oil. mass: 247 (M+1)⁺.

### Reference Example 1-20

### 3-cyclopropyl-2-oxopropanoic acid ethyl ester

In acetonitrile (120 mL) and water (30 mL) was suspended N-bromosuccinimide (22.5 g). To the suspension was then dropwise added a solution of 2-(cyclopropylmethyl)-1,3-dithiane-2-carboxylic acid ethyl ester (5.20 g) obtained in Reference Example 1-19 in acetonitrile (15.0 mL) at 0° C., and the mixture was stirred at room temperature for 1 hour. The reaction solution was extracted with hexane/dichloromethane (1:1), and the organic layer was washed with a saturated aqueous sodium sulfite solution and then with a saturated saline solution, and dried over anhydrous sodium sulfate. Insoluble matters were filtered out, and the filtrate was then concentrated under reduced pressure. Chloroform was added to the residue, insoluble matters were filtered out, and the filtrate was then concentrated under reduced pressure to give the title compound (2.95 g) as a pale yellow oil. mass: 157 (M+1)⁺.

### Reference Example 1-21

### 3-bromo-3-cyclopropyl-2-oxopropanoic acid ethyl ester

The title compound was obtained as a colorless oil by the same method as in Reference Example 1-14, methods equivalent thereto or combinations of these with usual methods using 3-cyclopropyl-2-oxopropanoic acid ethyl ester obtained in Reference Example 1-20 instead of 2-oxobutanoic acid ethyl ester obtained in Reference Example 1-13. mass: 235 (M+1)⁺.

### Reference Example 1-22

### 5-cyclopropyl-2-thioxo-2,3-dihydro-1,3-thiazol-4-carboxylic acid ethyl ester

The title compound was obtained as a white solid by the same method as in Reference Example 1-15, methods equivalent thereto or combinations of these with usual methods using 3-bromo-3-cyclopropyl-2-oxopropanoic acid ethyl ester obtained in Reference Example 1-21 instead of 3-bromo-2-oxobutanoic acid ethyl ester obtained in Reference Example 1-14. mass: 230 (M+1)⁺.

### Reference Example 2-1

### 4-hydroxypyridin-2,6-dicarboxylic acid diethyl ester

In ethanol (60.0 L) was dissolved 4-hydroxypyridin-2,6-carboxylic acid hydrate (3.00 kg). To the solution was added p-toluenesulfonic acid monohydrate (514 g), and the mixture was heated to reflux overnight. The reaction solution was concentrated under reduced pressure, a saturated aqueous sodium hydrogencarbonate solution was added to the residue, and the mixture was extracted with chloroform. The organic layer was washed with a saturated saline solution and dried over anhydrous sodium sulfate. Insoluble matters were filtered out, and the filtrate was concentrated under reduced pressure to give the title compound (3.48 kg) as a yellow oil. mass: 240 (M+1)⁺.

### Reference Example 2-2

### 4-(benzyloxy)pyridin-2,6-dicarboxylic acid diethyl ester

In N,N-dimethylformamide (20.0 L) was dissolved 4-hydroxypyridin-2,6-dicarboxylic acid diethyl ester (3.48 kg) obtained in Reference Example 2-1. To the solution were added potassium carbonate (2.01 kg) and benzyl bromide (1.73 L), and the mixture was stirred at room temperature for 4 hours. Water was added to the reaction solution, and the mixture was extracted with ethyl acetate. The organic layer was washed with a saturated saline solution and dried over anhydrous sodium sulfate. Insoluble matters were filtered out, the filtrate was concentrated under reduced pressure, and the resultant residue was recrystallized from heptane-ethyl acetate to give the title compound (3.56 kg) as a white solid. mass: 330 (M+1)⁺.

### Reference Example 2-3

### 4-(benzyloxy)-6-(hydroxymethyl)pyridin-2-carboxylic acid ethyl ester

In ethanol (8.90 L) was dissolved 4-(benzyloxy)pyridin-2,6-dicarboxylic acid diethyl ester (1.78 kg) obtained in Reference Example 2-2. To the solution were added calcium chloride (420 g) and sodium borohydride (140 g) at 0° C., and the mixture was stirred at room temperature for 5 hours. To the reaction solution was added 5N hydrochloric acid, and the mixture was concentrated under reduced pressure. To the residue was added 1N hydrochloric acid, and the mixture was extracted with chloroform. The organic layer was washed with water, a saturated aqueous sodium hydrogen-carbonate solution and a saturation saline solution sequentially and dried over anhydrous sodium sulfate. Insoluble matters were filtered out, and the filtrate was concentrated under reduced pressure. The resultant residue was recrystallized from heptane to give the title compound (1.09 kg) as a white solid. mass: 288 (M+1)⁺.

### Reference Example 2-4

### 4-(benzyloxy)-6-[(tetrahydro-2H-pyran-2-yloxy)methyl]pyridin-2-carboxylic acid ethyl ester

In chloroform (1,500 mL) was dissolved 4-(benzyloxy)-6-(hydroxymethyl)-pyridin-2-carboxylic acid ethyl ester (300 g) obtained in Reference Example 2-3. To the solution were added 3,4-dihydro-2H-pyran (191 mL) and p-toluenesulfonic acid pyridinium (26.2 g), and the mixture was heated to reflux for 3 hours. A saturated aqueous sodium hydrogencarbonate solution was added to the reaction solution, and the mixture was extracted with chloroform. The organic layer was washed with a saturated saline solution and dried over anhydrous sodium sulfate. Insoluble matters were filtered out, and the filtrate was concentrated under reduced pressure to give the title compound (388 g) as a yellow oil. mass: 372 (M+1)⁺.

### Reference Example 2-5

### 4-hydroxy-6-[(tetrahydro-2H-pyran-2-yloxy)methyl]pyridin-2-carboxylic acid ethyl ester

In methanol (1,900 mL) was dissolved 4-(benzyloxy)-6-[(tetrahydro-2H-pyran-2-yloxy)methyl]pyridin-2-carboxylic acid ethyl ester (388 g) obtained in Reference Example 2-4. To the solution were added 10% palladium carbon (77.6 g) and cyclohexene (1,060 mL), and the mixture was stirred at 80° C. for 1 hour. The reaction solution was Celite™ filtered, and the filtrate was concentrated under reduced pressure to give the title compound (298 g) as a yellow oil. mass: 282 (M+1)⁺.

### Reference Example 2-6

### 6-[(tetrahydro-2H-pyran-2-yloxy)methyl]-4-{[(trifluoromethyl)sulfonyl]oxy}pyridin-2-carboxylic acid ethyl ester

In chloroform (1,250 mL) were dissolved 4-hydroxy-6-[(tetrahydro-2H-pyran-2-yloxy)methyl]pyridin-2-carboxylic acid ethyl ester (250 g) obtained in Reference Example 2-5 and triethylamine (149 mL). To the solution was slowly added trifluoromethanesulfonic anhydride (165 mL) at 0° C., and the mixture was stirred at the same temperature for 1 hour. A saturated aqueous sodium hydrogencarbonate solution was added to the reaction solution, and the mixture was extracted with chloroform. The organic layer was washed with a saturated saline solution and dried over anhydrous sodium sulfate. Insoluble matters were filtered out, the filtrate was concentrated under reduced pressure, and the resultant residue was purified by NH silica gel column chromatography (chloroform) to yield the title compound (364 g) as a yellow oil. mass: 414 (M+1)⁺.

### Reference Example 2-7

### 4-(benzyloxy)-6-[(tetrahydro-2H-pyran-2-yloxy)methyl]pyridin-2-carboxylic acid

In methanol (8.30 L) was dissolved 4-(benzyloxy)-6-[(tetrahydro-2H-pyran-2-yloxy)methyl]pyridin-2-carboxylic acid ethyl ester (2.07 kg) obtained in Reference Example 2-4. To the solution was added a 2N aqueous sodium hydroxide solution (4.20 L) at 0° C., and the mixture was stirred at room temperature for 1 hour. The reaction solution was concentrated under reduced pressure, water was added to the residue, and the mixture was extracted with tert-butyl-methylether. A water layer was adjusted to pH 4 with 5N hydrochloric acid and extracted with chloroform, and the organic layer was then washed with a saturated saline solution and dried over anhydrous sodium sulfate. Insoluble matters were filtered out, and the filtrate was concentrated under reduced pressure to give the title compound (2.04 kg) as a yellow oil. mass: 342 (M−1)⁺.

### Reference Example 2-8

{4-(benzyloxy)-6-1(tetrahydro-2H-pyran-2-yloxy)methyl]pyridin-2-yl}carbamic acid tert-butyl ester

In dioxane (8.20 L) were dissolved 4-(benzyloxy)-6-[(tetrahydro-2H-pyran-2-yloxy)methyl]pyridin-2-carboxylic acid (1.02 kg) obtained in Reference Example 2-7, triethylamine (620 mL) and tert-butanol (2.80 L). To the solution was slowly added diphenylphosphoryl azide (610 mL) at 80° C., and the mixture was stirred at the same temperature for 1 hour. Water was added to the reaction solution, and the mixture was extracted with tert-butylmethylether. The organic layer was washed with water and a saturated saline solution and dried over anhydrous sodium sulfate. Insoluble matters were filtered out, the filtrate was concentrated under reduced pressure, and the resultant residue was recrystallized from tert-butylmethylether-heptane to give the title compound (690 g) as a white solid. mass: 415 (M+1)⁺.

### Reference Example 2-9

{4-hydroxy-6-[(tetrahydro-2H-pyran-2-yloxy)methyl]pyridin-2-yl}carbamic acid tert-butyl ester

The title compound was obtained by the same method as in Reference Example 2-5, methods equivalent thereto or combinations of these with usual methods by replacing 4-(benzyloxy)-6-[(tetrahydro-2H-pyran-2-yloxy)methyl]pyridin-2-carboxylic acid ethyl ester obtained in Reference Example 2-4 by {4-(benzyloxy)-6-[(tetrahydro-2H-pyran-2-yloxy)methyl]pyridin-2-yl}carbamic acid tert-butyl ester obtained in Reference Example 2-8. mass: 325 (M+1)⁺.

### Reference Example 2-10

2-[(tert-butoxycarbonyl)amino]-6-[(tetrahydro-2H-pyran-2-yloxy)methyl]pyridin-4-yl trifluoromethanesulfonic acid

The title compound was obtained as a pale yellow oil by the same method as in Reference Example 2-6, methods equivalent thereto or combinations of these with usual methods by replacing 4-hydroxy-6-[(tetrahydro-2H-pyran-2-yloxy)methyl]pyridin-2-carboxylic acid ethyl ester obtained in Reference Example 2-5 by {4-hydroxy-6-[(tetrahydro-2H-pyran-2-yloxy)methyl]pyridin-2-yl}carbamic acid tert-butyl ester obtained in Reference Example 2-9. mass: 457 (M+1)⁺.

### Reference Example 2-11

### {4-(morpholin-4-yl)-6-[(tetrahydro-2H-pyran-2-yloxy)methyl]pyridin-2-yl}carbamic acid tert-butyl ester

In dimethylsulfoxide (15.0 mL) was dissolved 2-[(tert-butoxycarbonyl)amino]-6-[(tetrahydro-2H-pyran-2-yloxy)methyl]pyridin-4-yl trifluoromethanesulfonic acid (1.02 g) obtained in Reference Example 2-10. To the solution was added morpholine (1.17 mL), and the mixture was stirred at 50° C. for 21 hours. An aqueous sodium hydrogencarbonate solution was added to the reaction solution, and the mixture was extracted with ethyl acetate. The organic layer was washed with an aqueous sodium hydrogencarbonate solution and a saturated saline solution and dried over anhydrous sodium sulfate. Insoluble matters were filtered out, the filtrate was concentrated under reduced pressure, and the resultant residue was purified by silica gel column chromatography (hexane/ethyl acetate=1:0 to 1:1) to yield the title compound (879 mg) as a colorless solid. mass: 394 (M+1)⁺.

### Reference Example 2-12

### [6-[(tetrahydro-2H-pyran-2-yloxy)methyl]-4-(thiomorpholin-4-yl)pyridin-2-yl]carbamic acid tert-butyl ester

The title compound was obtained by the same method as in Reference Example 2-11, methods equivalent thereto or combinations of these with usual methods using thiomorpholine instead of morpholine. mass: 410 (M+1)⁺.

### Reference Example 2-13

### 4-(4,4-difluoropiperidin-1-yl)-6-[(tetrahydro-2H-pyran-2-yloxy)methyl]pyridin-2-carboxylic acid ethyl ester

In N,N-dimethylformamide (900 mL) was dissolved 4,4-difluoropiperidine hydrochloride (111 g). To the solution were added 335 mL of N,N-diisopropylethylamine and 1,300 g of molecular sieve 4A (powder), and the mixture was stirred at room temperature for 1 hour. To the reaction solution was added a solution of 6-[(tetrahydro-2H-pyran-2-yloxy)methyl]-4-{[(trifluoromethyl)-sulfonyl]oxy}-2-pyridinecarboxylic acid ethyl ester (264 g) obtained in Reference Example 2-6 in N,N-dimethylformamide (400 mL), and the mixture was stirred overnight at 100° C. The reaction solution was Celite™ filtered, and the filtrate was diluted with ethyl acetate. This solution was washed with water and a saturated saline solution and dried over anhydrous sodium sulfate. Insoluble matters were filtered out, the filtrate was concentrated under reduced pressure, and the resultant residue was purified by NH silica gel column chromatography (hexane/ethyl acetate=1:1) to yield the title compound (241 g) as a yellow oil. mass: 385(M+1)⁺.

### Reference Example 2-14

### 4-(4,4-difluoropiperidin-1-yl)-6-[(tetrahydro-2H-pyran-2-yloxy)methyl]pyridin-2-pyridinecarboxylic acid

In methanol (940 mL) was dissolved 4-(4,4-difluoropiperidin-1-yl)-6-[(tetrahydro-2H-pyran-2-yloxy)methyl]pyridin-2-carboxylic acid ethyl ester (241 g) obtained in Reference Example 2-13. To the solution was added a 2N aqueous sodium hydroxide solution (470 mL) at 0° C., and the mixture was stirred at room temperature for 1 hour. The reaction solution was concentrated under reduced pressure, water was added to the resultant residue, and the mixture was extracted with diethyl ether. The water layer was adjusted to pH 4 with 5N hydrochloric acid and was extracted with chloroform. The organic layer was washed with a saturated saline solution and dried over anhydrous sodium sulfate. Insoluble matters were filtered out, and the filtrate was concentrated under reduced pressure to give the title compound (199 g) as a yellow oil. mass: 357 (M+1)⁺.

### Reference Example 2-15

### {4-(4,4-difluoropiperidin-1-yl)-6-[(tetrahydro-2H-pyran-2-yloxy)methyl]pyridin-2-yl}carbamic acid tert-butyl ester

In dioxane (1,600 mL) were dissolved 4-(4,4-difluoropiperidin-1-yl)-6-[(tetrahydro-2H-pyran-2-yloxy)methyl]pyridin-2-pyridinecarboxylic acid (199 g) obtained in Reference Example 2-14, triethylamine (117 mL) and tert-butanol (534 mL). To the solution was slowly added diphenylphosphoryl azide (133 mL) at 80° C., and the mixture was stirred at the same temperature for 2 hours. Water was added to the reaction solution, the mixture was extracted with ethyl acetate, and the organic layer was then washed with water and a saturated saline solution and dried over anhydrous sodium sulfate. Insoluble matters were filtered out, the filtrate was concentrated under reduced pressure, and the resultant residue was purified by NH silica gel column chromatography (hexane/ethyl acetate=1:1) to yield the title compound (220 g) as a yellow oil. mass: 428 (M+1)⁺.

### Reference Example 2-16

### {4-(4-fluoropiperidin-1-yl)-6-[(tetrahydro-2H-pyran-2-yloxy)methyl]pyridin-2-yl}carbamic acid tert-butyl ester

The title compound was obtained as a pale yellow oil by the same methods as in Reference Examples 2-13 to 2-15, methods equivalent thereto or combinations of these with usual methods using 4-fluoropiperidine hydrochloride instead of 4,4-difluoropiperidine hydrochloride. mass: 410 (M+1)⁺.

### Reference Example 2-17

### {4-[(3-exo)-3-fluoro-8-azabicyclo[3,2,1]octo-8-yl]-6-[(tetrahydro-2H-pyran-2-yloxy)methyl]pyridin-2-yl}carbamic acid tert-butyl ester

The title compound was obtained as a yellow oil by the same methods as in Reference Examples 2-13 to 2-15, methods equivalent thereto or combinations of these with usual methods using (3-exo)-3-fluoro-8-azabicyclo[3,2,1]octane hydrochloride (synthesis method: J. Org. Chem., 2002, 67, 8970) instead of 4,4-difluoropiperidine hydrochloride. mass: 436 (M+1)⁺.

### Reference Example 2-18

### {4-(3,3-difluoro-8-azabicyclo[3,2,1]octo-8-yl)-6-[(tetrahydro-2H-pyran-2-yloxy)methyl]pyridin-2-yl}carbamic acid tert-butyl ester

The title compound was obtained as a yellow oil by the same methods as in Reference Examples 2-13 to 2-15, methods equivalent thereto or combinations of these with usual methods using 3,3-difluoro-8-azabicyclo[3,2,1]octane hydrochloride obtained in Reference Example 1-4 instead of 4,4-difluoropiperidine hydrochloride. mass: 454 (M+1)⁺.

### Reference Example 2-19

### [6-(hydroxymethyl)-4-(thiomorpholin-4-yl)pyridin-2-yl]carbamic acid tert-butyl ester

In ethanol (4.00 mL) was dissolved [6-[(tetrahydro-2H-pyran-2-yloxy)methyl]-4-(thiomorpholin-4-yl)pyridin-2-yl]carbamic acid tert-butyl ester (400 mg) obtained in Reference Example 2-12. To the solution was added p-toluenesulfonic acid monohydrate (223 mg) at room temperature, and the mixture was stirred overnight. A saturated aqueous sodium hydrogencarbonate solution was added to the reaction solution, and the mixture was extracted with chloroform. The organic layer was washed with a saturated saline solution and then dried over magnesium sulfate. Insoluble matters were filtered out, the filtrate was concentrated under reduced pressure, and the residue was purified by silica gel column chromatography (hexane/ethyl acetate=98:2 to 30:70) to yield the title compound (318 mg) as a colorless oil. mass: 326 (M+1)⁺.

### Reference Example 2-20

### [6-(tert-butoxycarbonyl)amino]-4-(thiomorpholin-4-yl)pyridin-2-yl]methyl methane sulfonate

In ethyl acetate (1.5 ml) was dissolved [6-(hydroxymethyl)-4-(thiomorpholin-4-yl)pyridin-2-yl]carbamic acid tert-butyl ester (150 mg) obtained in Reference Example 2-19. To the solution were added triethylamine (128 μL) and methanesulphonyl chloride (72.0 μL) under ice-cooling, and the mixture was stirred at the same temperature for 30 minutes. A saturated saline solution was added to the reaction solution, and the mixture was extracted with ethyl acetate. The organic layer was washed with a saturated saline solution and dried over magnesium sulfate. Insoluble matters were filtered out, and the filtrate was concentrated under reduced pressure to give the title compound (180 mg) as a colorless oil. mass: 404 (M+1)⁺.

### Reference Example 2-21

### {6-[(tert-butoxycarbonyl)amino]-4-(4,4-difluoropiperidin-1-yl)pyridin-2-yl}methyl methane sulfonate

The title compound was obtained as a yellow oil by the same methods as in Reference Examples 2-19 and 2-20, methods equivalent thereto or combinations of these with usual methods using {4-(4,4-difluoropiperidin-1-yl)-6-[(tetrahydro-2H-pyran-2-yloxy)methyl]pyridin-2-yl}carbamic acid tert-butyl ester obtained in Reference Example 2-15 instead of [6-[(tetrahydro-2H-pyran-2-yloxy)methyl]-4-(thiomorpholin-4-yl)pyridin-2-yl]carbamic acid tert-butyl ester obtained in Reference Example 2-12. mass: 422 (M+1)⁺.

### Reference Example 2-22

### {6-[(tert-butoxycarbonyl)amino]-4-(4-fluoropiperidin-1-yl)pyridin-2-yl}methyl methane sulfonate

The title compound was obtained as a yellow oil by the same methods as in Reference Examples 2-19 and 2-20, methods equivalent thereto or combinations of these with usual methods using {4-(4-fluoro-1-piperidinyl)-6-[(tetrahydro-2H-pyran-2-yloxy)methyl]pyridin-2-yl}carbamic acid tert-butyl ester obtained in Reference Example 2-16 instead of [6-[(tetrahydro-2H-pyran-2-yloxy)methyl]-4-(thio-morpholin-4-yl)pyridin-2-yl]carbamic acid tert-butyl ester obtained in Reference Example 2-12. mass: 404 (M+1)⁺.

### Reference Example 2-23

### {6-[(tert-butoxycarbonyl)amino]-4-[(3-exo)-3-fluoro-8-azabicyclo[3,2,1]octo-8-yl]pyridin-2-yl}methyl methane sulfonate

The title compound was obtained as a yellow oil by the same methods as in Reference Examples 2-19 and 2-20, methods equivalent thereto or combinations of these with usual methods using {4-[(3-exo)-3-fluoro-8-azabicyclo[3,2,1]octo-8-yl]-6-[(tetrahydro-2H-pyran-2-yloxy)methyl]pyridin-2-yl}carbamic acid tert-butyl ester obtained in Reference Example 2-17 instead of [6-[(tetrahydro-2H-pyran-2-yloxy)-methyl]-4-(thiomorpholin-4-yl)pyridin-2-yl]carbamic acid tert-butyl ester obtained in Reference Example 2-12. mass: 430 (M+1)⁺.

### Reference Example 2-24

### {6-[(tert-butoxycarbonyl)amino]-4-(3,3-difluoro-8-azabicyclo[3,2,1]octo-8-yl)pyridin-2-yl}methyl methane sulfonate

The title compound was obtained as a yellow oil by the same methods as in Reference Examples 2-19 and 2-20, methods equivalent thereto or combinations of these with usual methods using {4-(3,3-difluoro-8-azabicyclo[3,2,1]-octo-8-yl)-6-[(tetrahydro-2H-pyran-2-yloxy)methyl]pyridin-2-yl}carbamic acid tert-butyl ester obtained in Reference Example 2-18 instead of [6-[(tetrahydro-2H-pyran-2-yloxy)-methyl]-4-(thiomorpholin-4-yl)pyridin-2-yl]carbamic acid tert-butyl ester obtained in Reference Example 2-12. mass: 448 (M+1)⁺.

### Reference Example 2-25

### [6-{[(4,5-dimethyl-1,3-oxazol-2-yl)sulfanyl]methyl}-4-(thiomorpholin-4-yl)pyridin-2-yl]carbamic acid tert-butyl ester

In dimethylformamide (1.50 mL) was dissolved [6-(tert-butoxycarbonyl)-amino]-4-(thiomorpholin-4-yl)pyridin-2-yl]methyl methane sulfonate (180 mg) obtained in Reference Example 2-20. To the solution were added potassium carbonate (127 mg) and 4,5-dimethyl-1,3-oxazol-2 (3H)-thione (89 mg) at room temperature, and the mixture was stirred for 2 hours. Water was added to the reaction solution, and the mixture was extracted with ethyl acetate. The organic layer was washed with a saturated saline solution and dried over magnesium sulfate. Insoluble matters were filtered out, the filtrate was concentrated under reduced pressure, and the resultant residue was purified by silica gel column chromatography (hexane/ethyl acetate=95:5 to 50:50) to yield the title compound (194 mg) as a yellow oil. mass: 437 (M+1)⁺.

### Reference Example 2-26

### [6-{1 (4,5-dimethyl-1,3-thiazol-2-yl)sulfanyl]methyl}-4-(thiomorpholin-4-yl)pyridin-2-yl]carbamic acid tert-butyl ester

The title compound was obtained as a yellow oil by the same method as in Reference Example 2-25, methods equivalent thereto or combinations of these with usual methods using 4,5-dimethyl-1,3-thiazol-2 (3H)-thione instead of 4,5-dimethyl-1,3-oxazol-2 (3H)-thione. mass: 453 (M+1)⁺.

### Reference Example 2-27

### [6-{[(5-ethyl-4-methyl-1,3-thiazol-2-yl)sulfanyl]methyl}-4-(4-fluoropiperidin-1-yl)pyridin-2-yl]carbamic acid tert-butyl ester

The title compound was obtained as a yellow oil by the same method as in Reference Example 2-25, methods equivalent thereto or combinations of these with usual methods using [6-(tert-butoxycarbonyl)amino]-4-(4-fluoropiperidin-1-yl)-pyridin-2-yl]methyl methane sulfonate obtained in Reference Example 2-22 instead of [6-(tert-butoxycarbonyl)amino]-4-(thiomorpholin-4-yl)pyridin-2-yl]methyl methane sulfonate obtained in Reference Example 2-20 and using 5-ethyl-4-methyl-1,3-thiazol-2 (3H)-thione instead of 4,5-dimethyl-1,3-oxazol-2 (3H)-thione. mass: 467 (M+1)⁺.

### Reference Example 2-28

### (6-{[(5-ethyl-4-methyl-1,3-thiazol-2-yl)sulfanyl]methyl}-4-[(3-exo)-3-fluoro-8-azabicyclo[3,2,1]octo-8-yl]pyridin-2-yl)carbamic acid tert-butyl ester

The title compound was obtained as a yellow oil by the same method as in Reference Example 2-25, methods equivalent thereto or combinations of these with usual methods using {6-[(tert-butoxycarbonyl)amino]-4-[(3-exo)-3-fluoro-8-azabicyclo[3,2,1]octo-8-yl]pyridin-2-yl}methyl methane sulfonate obtained in Reference Example 2-23 instead of [6-(tert-butoxycarbonyl)amino]-4-(thiomorpholin-4-thiomorpholinyl)pyridin-2-yl]methyl methane sulfonate obtained in Reference Example 2-20 and using 5-ethyl-4-methyl-1,3-thiazol-2 (3H)-thione instead of 4,5-dimethyl-1,3-oxazol-2 (3H)-thione. mass: 493 (M+1)⁺.

### Reference Example 2-29

### [4-(3,3-difluoro-8-azabicyclo[3,2,1]octo-8-yl)-6-{[(1,5-dimethyl-1H-pyrazol-3-yl)sulfanyl]methyl}pyridin-2-yl)carbamic acid tert-butyl ester

The title compound was obtained as a yellow oil by the same method as in Reference Example 2-25, methods equivalent thereto or combinations of these with usual methods using {6-[(tert-butoxycarbonyl)amino]-4-(3,3-difluoro-8-azabicyclo[3,2,1]octo-8-yl)pyridin-2-yl]methyl methane sulfonate obtained in Reference Example 2-24 instead of [6-(tert-butoxycarbonyl)amino]-4-(thio-morpholin-4-yl)-pyridin-2-yl]methyl methane sulfonate obtained in Reference Example 2-20 and using 1,5-dimethyl-1,2-dihydro-3H-pyrazol-3-thione obtained in Reference Example 1-7 instead of 4,5-dimethyl-1,3-oxazol-2 (3H)-thione. mass: 480(M+1)⁺.

### Reference Example 2-30

### [4-(4,4-difluoropiperidin-1-yl)-6-[(5-ethyl-1H-pyrazol-3-yl)sulfanyl]methyl}pyridin-2-yl]carbamic acid tert-butyl ester

In dimethylformamide (2.00 mL) was dissolved {6-[(tert-butoxycarbonyl)-amino]-4-(4,4-difluoro-piperidin-1-yl)-pyridin-2-yl]methyl methane sulfonate (144 mg) obtained in Reference Example 2-21. To the solution was added diazabicycloundecene (0.132 mL) at room temperature. The mixture was stirred for 30 minutes, and 5-ethyl-1,2-dihydro-3H-pyrazol-3-thione hydrochloride (148 mg) obtained in Reference Example 1-9 and potassium carbonate (145 mg) were then added at room temperature. The mixture was stirred overnight. Water was added to the reaction solution, the mixture was extracted with ethyl acetate, and the organic layer was washed with water and a saturated saline solution and then dried over anhydrous sodium sulfate. Insoluble matters were filtered out, and the filtrate was concentrated under reduced pressure. The resultant residue was purified by silica gel column chromatography (hexane/ethyl acetate=75:25 to 50:50) to yield the title compound (160 mg) as a pale yellow oil. mass: 454 (M+1)⁺.

### Reference Example 2-31

### [4-(4,4-difluoropiperidin-1-yl)-6-{[(5-ethyl-1-methyl-1H-pyrazol-3-yl)sulfanyl]methyl}pyridin-2-yl]carbamic acid tert-butyl ester

In dimethylformamide (2.00 mL) was dissolved [4-(4,4-difluoropiperidin-1-yl)-6-{[(5-ethyl-1H-pyrazol-3-yl)sulfanyl]methyl}pyridin-2-yl]carbamic acid tert-butyl ester (160 mg) obtained in Reference Example 2-30. To the solution were added cesium carbonate (345 mg) and methyl iodide (33.1 μL) at room temperature, and the mixture was stirred overnight. Water was added to the reaction solution, the mixture was extracted with ethyl acetate, and the organic layer was washed with water and a saturated saline solution and then dried over anhydrous sodium sulfate. Insoluble matters were filtered out, and the filtrate was concentrated under reduced pressure. The resultant residue was purified by silica gel column chromatography (hexane/ethyl acetate=75:25 to 34:66) to yield the title compound (27.4 mg) as a colorless oil. mass: 468 (M+1)⁺.

### Reference Example 2-32

### [4-(4,4-difluoropiperidin-1-yl)-6-{[(5-ethyl-1,4-dimethyl-1H-pyrazol-3-yl)sulfanyl]methyl}pyridin-2-yl]carbamic acid tert-butyl ester

The title compound was obtained as a pale yellow oil by the same methods as in Reference Examples 2-30 and 2-31, methods equivalent thereto or combinations of these with usual methods using 5-ethyl-4-methyl-1,2-dihydro-3H-pyrazol-3-thione hydrochloride obtained in Reference Example 1-12 instead of 5-ethyl-1,2-dihydro-3H-pyrazol-3-thione hydrochloride obtained in Reference Example 1-9. mass: 482 (M+1)⁺.

### Reference Example 2-33

### [6-{[(4,5-dimethyl-1,3-oxazol-2-yl)sulfanyl]methyl}-4-(thiomorpholin-4-yl)pyridin-2-yl][(6-fluoropyridin-2-yl)methyl]carbamic acid tert-butyl ester

In dimethylformamide (1.50 mL) was dissolved [6-{[(4,5-dimethyl-1,3-oxazol-2-yl)-sulfanyl]methyl}-4-(thiomorpholin-4-yl)pyridin-2-yl]carbamic acid tert-butyl ester (83.0 mg) obtained in Reference Example 2-25. To the solution was added sodium hydride (23.0 mg) at room temperature, and the mixture was stirred for 30 minutes. To the reaction solution was added 2-(chloromethyl)-6-fluoropyridine (138 mg), and the mixture was stirred for 30 hours. A saturated aqueous ammonium chloride solution was added to the reaction solution, and the mixture was extracted with ethyl acetate. The organic layer was dried over magnesium sulfate and then washed with a saturated saline solution. Insoluble matters were filtered out, and the filtrate was concentrated under reduced pressure. The resultant residue was purified by silica gel column chromatography (hexane/ethyl acetate=95:5 to 25:75) to yield the title compound (51.3 mg) as a yellow oil. mass: 546 (M+1)⁺.

### Reference Example 2-34

### [6-{[(4,5-dimethyl-1,3-thiazol-2-yl)sulfanyl]methyl}-4-(thiomorpholin-4-yl)pyridin-2-yl][(6-methylpyridin-2-yl)methyl]carbamic acid tert-butyl ester

The title compound was obtained as a yellow oil by the same method as in Reference Example 2-33, methods equivalent thereto or combinations of these with usual methods using [6-{[(4,5-dimethyl-1,3-thiazol-2-yl)-sulfanyl]methyl}-4-(thiomorpholin-4-yl)pyridin-2-yl]carbamic acid tert-butyl ester obtained in Reference Example 2-26 instead of [6-{[(4,5-dimethyl-1,3-oxazol-2-yl)-sulfanyl]methyl}-4-(thiomorpholin-4-yl)pyridin-2-yl]carbamic acid tert-butyl ester obtained in Reference Example 2-25 and using 2-(chloromethyl)-6-methylpyridine hydrochloride instead of 2-(chloromethyl)-6-fluoropyridine. mass: 558 (M+1)⁺.

### Reference Example 2-35

### [6-{1 (5-ethyl-4-methyl-1,3-thiazol-2-yl)sulfanyl]methyl}-4-(4-fluoropiperidin-1-yl)pyridin-2-yl][(6-methylpyridin-2-yl)methyl]carbamic acid tert-butyl ester

The title compound was obtained as a yellow oil by the same method as in Reference Example 2-33, methods equivalent thereto or combinations of these with usual methods using [6-{[(5-ethyl-4-methyl-1,3-thiazol-2-yl)-sulfanyl]-methyl)-4-(4-fluoro-piperidin-1-yl)pyridin-2-yl]carbamic acid tert-butyl ester obtained in Reference Example 2-27 instead of [6-{[(4,5-dimethyl-1,3-oxazol-2-yl)sulfanyl]-methyl}-4-(thiomorpholin-4-yl)pyridin-2-yl]carbamic acid tert-butyl ester obtained in Reference Example 2-25 and using 2-(chloromethyl)-6-methylpyridine hydrochloride instead of 2-(chloromethyl)-6-fluoropyridine. mass: 572 (M+1)⁺.

### Reference Example 2-36

### (6-{[(5-ethyl-4-methyl-1,3-thiazol-2-yl)sulfanyl]methyl}-4-[(3-exo)-3-fluoro-8-azabicyclo[3,2,1]octo-8-yl]pyridin-2-yl)-2-propen-1-ylcarbamic acid tert-butyl ester

The title compound was obtained as a yellow oil by the same method as in Reference Example 2-33, methods equivalent thereto or combinations of these with usual methods using (6-{[(5-ethyl-4-methyl-1,3-thiazol-2-yl)sulfanyl]-methyl}-4-[(3-exo)-3-fluoro-8-azabicyclo[3,2,1]octo-8-yl]pyridin-2-yl)carbamic acid tert-butyl ester obtained in Reference Example 2-28 instead of [6-{[(4,5-dimethyl-1,3-oxazol-2-yl)sulfanyl]methyl}-4-(thiomorpholin-4-yl)pyridin-2-yl]carbamic acid tert-butyl ester obtained in Reference Example 2-25 and using allyl bromide instead of 2-(chloromethyl)-6-fluoropyridine. mass: 533 (M+1)⁺.

### Reference Example 2-37

### [4-(3,3-difluoro-8-azabicyclo[3,2,1]octo-8-yl)-6-{[(1,5-dimethyl-1H-pyrazol-3-yl)sulfanyl]methyl}pyridin-2-yl][(6-methylpyridin-2-yl)methyl]carbamic acid tert-butyl ester

The title compound was obtained as a yellow oil by the same method as in Reference Example 2-33, methods equivalent thereto or combinations of these with usual methods using [4-(3,3-difluoro-8-azabicyclo[3,2,1]octo-8-yl)-6-{[(1,5-dimethyl-1H-pyrazol-3-yl)sulfanyl]methyl}pyridin-2-yl)carbamic acid tert-butyl ester obtained in Reference Example 2-29 instead of [6-{[(4,5-dimethyl-1,3-oxazol-2-yl)sulfanyl]-methyl}-4-(thiomorpholin-4-yl)pyridin-2-yl]carbamic acid tert-butyl ester obtained in Reference Example 2-25 and using 2-(chloromethyl)-6-methylpyridine hydrochloride instead of 2-(chloromethyl)-6-fluoropyridine. mass: 585 (M+1)⁺.

### Reference Example 2-38

### [4-(4,4-difluoropiperidin-1-yl)-6-{[(5-ethyl-1-methyl-1H-pyrazol-3-yl)sulfanyl]methylpyridin-2-yl]prop-2-en-1-yl carbamic acid tert-butyl ester

The title compound was obtained as a yellow oil by the same method as in Reference Example 2-33, methods equivalent thereto or combinations of these with usual methods using [4-(4,4-difluoropiperidin-1-yl)-6-{[(5-ethyl-1-methyl-1H-pyrazol-3-yl)sulfanyl]methyl}pyridin-2-yl]carbamic acid tert-butyl ester obtained in Reference Example 2-31 instead of [6-{[(4,5-dimethyl-1,3-oxazol-2-yl)sulfanyl]-methyl}-4-(thiomorpholin-4-yl)pyridin-2-yl]carbamic acid tert-butyl ester obtained in Reference Example 2-25 and using allyl bromide instead of 2-(chloromethyl)-6-fluoropyridine. mass: 508 (M+1)⁺.

Compounds in Reference Examples 2-39 and 2-40 were obtained by the same method as in Reference Example 2-33, methods equivalent thereto or combinations of these with usual methods using [4-(4,4-difluoropiperidin-1-yl)-6-{[(5-ethyl-1,4-dimethyl-1H-pyrazol-3-yl)sulfanyl]methyl}pyridin-2-yl]carbamic acid tert-butyl ester obtained in Reference Example 2-32 instead of [6-{[(4,5-dimethyl-1,3-oxazol-2-yl)-sulfanyl]methyl}-4-(thiomorpholin-4-yl)pyridin-2-yl]carbamic acid tert-butyl ester obtained in Reference Example 2-25 and using the corresponding alkyl halides instead of 2-(chloromethyl)-6-fluoropyridine.

### Reference Example 2-39

### butyl[4-(4,4-difluoropiperidin-1-yl)-6-{[(5-ethyl-1,4-dimethyl-1H-pyrazol-3-yl)sulfanyl]methyl}pyridin-2-yl]carbamic acid tert-butyl ester

The title compound was obtained as a pale yellow oil by the same method as in Reference Example 2-33, methods equivalent thereto or combinations of these with usual methods using butyl bromide instead of 2-(chloromethyl)-6-fluoropyridine. mass: 538 (M+1)⁺.

### Reference Example 2-40

### [4-(4,4-difluoropiperidin-1-yl)-6-{[(5-ethyl-1,4-dimethyl-1H-pyrazol-3-yl)sulfanyl]methyl}pyridin-2-yl](2-methoxyethyl)carbamic acid tert-butyl ester

The title compound was obtained as a pale yellow oil by the same method as in Reference Example 2-33, methods equivalent thereto or combinations of these with usual methods using 2-methoxyethyl bromide instead of 2-(chloromethyl)-6-fluoropyridine. mass: 540 (M+1)⁺.

### Reference Example 2-41

### [(6-methylpyridin-2-yl)methyl]{4-(morpholin-4-yl)-6-[(tetrahydro-2H-pyran-2-yloxy)methyl]pyridin-2-yl}carbamic acid tert-butyl ester

The title compound was obtained as a yellow oil by the same method as in Reference Example 2-33, methods equivalent thereto or combinations of these with usual methods using {4-(morpholin-4-yl)-6-[(tetrahydro-2H-pyran-2-yloxy)methyl]-pyridin-2-yl}carbamic acid tert-butyl ester obtained in Reference Example 2-11 instead of [6-{[(4,5-dimethyl-1,3-oxazol-2-yl)sulfanyl]methyl}-4-(thiomorpholin-4-yl)-pyridin-2-yl]carbamic acid tert-butyl ester obtained in Reference Example 2-25 and using 2-(chloromethyl)-6-methylpyridine hydrochloride instead of 2-(chloromethyl)-6-fluoropyridine. mass: 499 (M+1)⁺.

### Reference Example 2-42

### [6-{(tert-butoxycarbonyl)[(6-methylpyridin-2-yl)methyl]amino}-4-(morpholin-4-yl)pyridin-2-yl]methyl methane sulfonate

The title compound was obtained as a colorless oil by the same methods as in Reference Examples 2-19 and 2-20, methods equivalent thereto or combinations of these with usual methods using [(6-methylpyridin-2-yl]methyl]{4-(morpholin-4-yl)-6-[(tetrahydro-2H-pyran-2-yloxy)methyl]pyridin-2-yl}carbamic acid tert-butyl ester obtained in Reference Example 2-41 instead of [6-[(tetrahydro-2H-pyran-2-yloxy)methyl]-4-(thiomorpholin-4-yl)-pyridin-2-yl]carbamic acid tert-butyl ester obtained in Reference Example 2-12. mass: 493 (M+1)⁺.

The compounds of Reference Examples 2-43 to 2-45 were obtained by the same method as in Reference Example 2-25, methods equivalent thereto or combinations of these with usual methods using [6-{(tert-butoxycarbonyl)-[(6-methylpyridin-2-yl)methyl]amino}-4-(morpholin-4-yl)pyridin-2-yl]methyl methane sulfonate obtained in Reference Example 2-42 instead of [6-(tert-butoxycarbonyl)-amino]-4-(thiomorpholin-4-yl)pyridin-2-yl]methyl methane sulfonate obtained in Reference Example 2-20 and using the corresponding 2-thioxo-2,3-dihydro-1,3-thiazol-4-carboxylic acid ethyl esters instead of 4,5-dimethyl-1,3-oxazol-2 (311)-thione.

### Reference Example 2-43

### 2-({[6-{(tert-butoxycarbonyl)[(6-methylpyridin-2-yl)methyl]amino}-4-(morpholin-4-yl)pyridin-2-yl]methyl}sulfanyl)-5-methyl-1,3-thiazol-4-carboxylic acid ethyl ester

The title compound was obtained as a pale yellow oil by the same method as in Reference Example 2-25, methods equivalent thereto or combinations of these with usual methods using 5-methyl-2-thioxo-2,3-dihydro-1,3-thiazol-4-carboxylic acid ethyl ester obtained in Reference Example 1-15 instead of 4,5-dimethyl-1,3-oxazol-2 (3H)-thione. mass: 600 (M+1)⁺.

### Reference Example 2-44

### 2-({[6-{(tert-butoxycarbonyl)[(6-methylpyridin-2-yl)methyl]amino-4-(morpholin-4-yl)pyridin-2-yl]methyl}sulfanyl)-5-ethyl-1,3-thiazol-4-carboxylic acid ethyl ester

The title compound was obtained as a pale yellow oil by the same method as in Reference Example 2-25, methods equivalent thereto or combinations of these with usual methods using 5-ethyl-2-thioxo-2,3-dihydro-1,3-thiazol-4-carboxylic acid ethyl ester obtained in Reference Example 1-18 instead of 4,5-dimethyl-1,3-oxazol-2 (3H)-thione. mass: 614(M+1)⁺.

### Reference Example 2-45

### 2-({[6-{(tert-butoxycarbonyl)[(6-methylpyridin-2-yl)methyl]amino}-4-(morpholin-4-yl)pyridin-2-yl]methyl}sulfanyl)-5-cyclopropyl-1,3-thiazol-4-carboxylic acid ethyl ester

The title compound was obtained as a pale yellow oil by the same method as in Reference Example 2-25, methods equivalent thereto or combinations of these with usual methods using 5-cyclopropyl-2-thioxo-2,3-dihydro-1,3-thiazol-4-carboxylic acid ethyl ester obtained in Reference Example 1-22 instead of 4,5-dimethyl-1,3-oxazol-2 (3H)-thione. mass: 626 (M+1)⁺.

### Reference Example 2-46

### [6-({[4-(hydroxymethyl)-5-methyl-1,3-thiazol-2-yl]sulfanyl}methyl)-4-(morpholin-4-yl)pyridin-2-yl][(6-methylpyridin-2-yl)methyl]carbamic acid tert-butyl ester

In tetrahydrofuran (3.00 mL) was dissolved 2-({[6-{(tert-butoxycarbonyl)-[(6-methylpyridin-2-yl)methyl]amino}-4-(morpholin-4-yl)pyridin-2-yl]methyl}sulfanyl)-5-methyl-1,3-thiazol-4-carboxylic acid ethyl ester (43.0 mg) obtained in Reference Example 2-43. To the solution was added lithium aluminum hydride (5.47 mg) at 0° C., and the mixture was stirred for 2 hours. An excessive amount of sodium sulfate decahydrate was added to the reaction solution, and the mixture was stirred at room temperature for 6 hours. Insoluble matters were filtered out, and the filtrate was then concentrated under reduced pressure to give the title compound (39.6 mg) as a colorless oil. mass: 558 (M+1)⁺.

### Reference Example 2-47

### [6-([5-ethyl-4-(hydroxymethyl)-1,3-thiazol-2-yl]sulfanyl}methyl)-4-(morpholin-4-yl)pyridin-2-yl][(6-methylpyridin-2-yl)methyl]carbamic acid tert-butyl ester

The title compound was obtained as a colorless oil by the same method as in Reference Example 2-46, methods equivalent thereto or combinations of these with usual methods using 2-({[6-{(tert-butoxycarbonyl)[(6-methylpyridin-2-yl)-methyl]amino}-4-(morpholin-4-yl)pyridin-2-yl]methyl}sulfanyl)-5-ethyl-1,3-thiazol-4-carboxylic acid ethyl ester obtained in Reference Example 2-44 instead of 2-({[6-{(tert-butoxycarbonyl)[(6-methylpyridin-2-yl)methyl]amino}-4-(morpholin-4-yl)pyridin-2-yl]methyl}sulfanyl)-5-methyl-1,3-thiazol-4-carboxylic acid ethyl ester obtained in Reference Example 2-43. mass: 572 (M+1)⁺.

### Reference Example 2-48

### [6-({[5-cyclopropyl-4-(hydroxymethyl)-1,3-thiazol-2-yl]sulfanyl}methyl)-4-(morpholin-4-yl)pyridin-2-yl][(6-methylpyridin-2-yl)methyl]carbamic acid tert-butyl ester

The title compound was obtained as a colorless oil by the same method as in Reference Example 2-46, methods equivalent thereto or combinations of these with usual methods using 2-({[6-{(tert-butoxycarbonyl)-[(6-methylpyridin-2-yl)methyl]-amino}-4-(morpholin-4-yl)pyridin-2-yl]methyl}sulfanyl)-5-cyclopropyl-1,3-thiazol-4-carboxylic acid ethyl ester obtained in Reference Example 2-45 instead of 2-({[6-{(tert-butoxycarbonyl)[(6-methylpyridin-2-yl)methyl]amino}-4-(morpholin-4-yl)pyridin-2-yl]methyl}sulfanyl)-5-methyl-1,3-thiazol-4-carboxylic acid ethyl ester obtained in Reference Example 2-43. mass: 584(M+1)⁺.

### Example 1

### 6-{[(4,5-dimethyl-1,3-oxazol-2-yl)sulfanyl]methyl}-N-[(6-fluoropyridin-2-yl)methyl]-4-(thiomorpholin-4-yl)pyridin-2-amine

Trifluoroacetic acid (1.00 mL) was added to [6-{[(4,5-dimethyl-1,3-oxazol-2-yl)-sulfanyl]methyl}-4-(thiomorpholin-4-yl)pyridin-2-yl][(6-fluoropyridin-2-yl)methyl]carbamic acid tert-butyl ester (50.2 mg) obtained in Reference Example 2-33, and the mixture was stirred at room temperature for 1 hour. The solvent was distilled off under reduced pressure. The resultant residue was purified by reversed-phase high-performance liquid column chromatography (acetonitrile/water=10:90 to 90:10) to yield the title compound (39.0 mg) as a brown oil.

¹H-NMR (CDCl₃) δ: 1.91 (3H, s), 2.12 (3H, s), 2.49 (4H, t, J=4.9 Hz), 3.57 (4H, t, J=4.9 Hz), 4.07 (2H, s), 4.37 (2H, d, J=5.9 Hz), 5.74 (1H, s), 6.21 (1H, s), 6.75 (1H, s), 6.95 (1H, dd, J=8.0, 2.5 Hz), 7.21 (1H, dd, J=8.0, 2.5 Hz), 7.85 (1 H, q, J=8.0 Hz). mass: 446 (M+1)⁺.

The compounds of Examples 2 to 8 were obtained by the same method as in Example 1, methods equivalent thereto or combinations of these with usual methods using the corresponding Boc protectors instead of [6-{[(4,5-dimethyl-1,3-oxazol-2-yl)-sulfanyl]methyl}-4-(thiomorpholin-4-yl)pyridin-2-yl][(6-fluoropyridin-2-yl)methyl]carbamic acid tert-butyl ester obtained in Reference Example 2-33.

### Example 2

### 6-{[(4,5-dimethyl-1,3-thiazol-2-yl)sulfanyl]methyl}-N-[(6-methylpyridin-2-yl)methyl]-4-(thiomorpholin-4-yl)pyridin-2-amine

The title compound was obtained by the same method as in Example 1, methods equivalent thereto or combinations of these with usual methods using [6-{[(4,5-dimethyl-1,3-thiazol-2-yl)sulfanyl]methyl}-4-(thiomorpholin-4-yl)pyridin-2-yl][(6-methylpyridin-2-yl)methyl]carbamic acid tert-butyl ester obtained in Reference Example 2-34.

¹H-NMR (CDCl₃) δ: 2.28 (6H, m), 2.55-2.58 (7H, m), 3.66 (4H, t, J=5.1 Hz), 4.22 (2H, s), 4.52 (2H, d, J=5.5 Hz), 5.43 (1H, brs), 5.59 (1H, d, J=2.0 Hz), 6.20 (1H, d, J=2.0 Hz), 7.03 (1H, d, J=7.8 Hz), 7.17 (1H, d, J=7.8 Hz), 7.53 (1H, t, J=7.6 Hz). mass: 458 (M+1)⁺.

### Example 3

### 6-{[(5-ethyl-4-methyl-1,3-thiazol-2-yl)sulfanyl]methyl}-4-(4-fluoropiperidin-1-yl)-N-[(6-methylpyridin-2-yl)methyl]pyridin-2-amine

The title compound was obtained by the same method as in Example 1, methods equivalent thereto or combinations of these with usual methods using [6-{[(5-ethyl-4-methyl-1,3-thiazol-2-yl)sulfanyl]methyl}-4-(4-fluoropiperidin-1-yl)pyridin-2-yl][(6-methylpyridin-2-yl)methyl]carbamic acid tert-butyl ester obtained in Reference Example 2-35.

¹H-NMR (CDCl₃) δ: 1.20 (3H, t, J=7.6 Hz), 1.85-1.90 (4H, m), 2.29 (3H, s), 2.55 (3H, s), 2.68 (2H, q, J=7.6 Hz), 3.23-3.43 (4H, m), 4.23 (2H, s), 4.53 (2H, d, J=5.9 Hz), 4.75-4.85 (1H, m), 5.42-5.44 (1H, m), 5.66 (1H, d, J=2.0 Hz), 6.27 (1H, d, J=2.0 Hz), 7.02 (1H, d, J=7.8 Hz), 7.17 (1H, d, J=7.8 Hz), 7.52 (1H, t, J=7.6 Hz). mass: 472 (M+1)⁺.

### Example 4

### 6-{[(5-ethyl-4-methyl-1,3-thiazol-2-yl)sulfanyl]methyl}-4-[(3-exo)-3-fluoro-8-azabicyclo[3,2,1]octo-8-yl]-N-(prop-2-en-1-yl)pyridin-2-amine

The title compound was obtained by the same method as in Example 1, methods equivalent thereto or combinations of these with usual methods using (6-[(5-ethyl-4-methyl-1,3-thiazol-2-yl)sulfanyl]methyl}-4-[(3-exo)-3-fluoro-8-azabicyclo[3,2,1]octo-8-yl]pyridin-2-yl)2-propen-1-ylcarbamic acid tert-butyl ester obtained in Reference Example 2-36.

¹H-NMR (CDCl₃) δ: 1.19 (3H, t, J=7.6 Hz), 1.67-1.82 (4H, m), 1.93-2.07 (4H, m), 2.29 (3H, s), 2.67 (2H, q, J=7.6 Hz), 3.85 (2H, t, J=5.7 Hz), 4.20-4.22 (4H, m), 4.57-4.64 (1H, m), 4.89-5.10 (1H, m), 5.13-5.17 (1H, m), 5.25-5.31 (1H, m), 5.53 (1H, d, J=2.0 Hz), 5.88-5.98 (1H, m), 6.17 (1H, d, J=2.0 Hz). mass: 433 (M+1)⁺.

### Example 5

### 4-(3,3-difluoro-8-azabicyclo[3,2,1]octo-8-yl)-6-{[(1,5-dimethyl-1H-pyrazol-3-yl)sulfanyl]methyl}-N-[(6-methylpyridin-2-yl)methyl]pyridin-2-amine

The title compound was obtained by the same method as in Example 1, methods equivalent thereto or combinations of these with usual methods using [4-(3,3-difluoro-8-azabicyclo[3,2,1]octo-8-yl)-6-{[(1,5-dimethyl-1H-pyrazol-3-yl)sulfanyl]methylpyridin-2-yl][(6-methylpyridin-2-yl)methyl]carbamic acid tert-butyl ester obtained in Reference Example 2-37.

¹H-NMR (CDCl₃) δ: 1.76-2.03 (8H, m), 2.15 (3H, s), 2.52 (3H, s), 3.68 (3H, s), 3.96 (2H, s), 4.16-4.18 (2H, brm), 4.48 (2H, d, J=5.9 Hz), 5.46 (1H, brs), 5.49 (1H, s), 5.93 (1H, s), 6.14 (1H, s), 6.99 (1H, d, J=7.4 Hz), 7.14 (1H, d, J=7.4 Hz), 7.49 (1H, t, J=7.4 Hz). mass: 485 (M+1)⁺.

### Example 6

### 4-(4,4-difluoropiperidin-1-yl)-6-{[(5-ethyl-1-methyl-1H-pyrazol-3-yl)sulfanyl]methyl}-N-(prop-2-en-1-yl)pyridin-2-amine

The title compound was obtained by the same method as in Example 1, methods equivalent thereto or combinations of these with usual methods using [4-(4,4-difluoropiperidin-1-yl)-6-{[(5-ethyl-1-methyl-1H-pyrazol-3-yl)sulfanyl]methyl}pyridin-2-yl]prop-2-en-1-yl carbamic acid tert-butyl ester obtained in Reference Example 2-38.

¹H-NMR (CDCl₃) δ: 1.22 (3H, t, J=7.4 Hz), 1.90-2.10 (4H, m), 2.55 (2H q, J=7.4 Hz), 3.45-3.50 (4H, m), 3.73 (3H, s), 3.84 (2H, s), 4.01 (2H, s), 4.62 (1H, brs), 5.18 (1H, dd, J=10.2, 1.5 Hz), 5.28 (1H, dd, J=17.1, 1.5 Hz), 5.56 (1H, d, J=2.2 Hz), 5.86-5.95 (1H m), 6.03 (1H, s), 6.32 (1H, d, J=2.2 Hz). mass: 408 (M+1)⁺.

### Example 7

### N-butyl-4-(4,4-difluoropiperidin-1-yl)-6-{[(5-ethyl-1,4-dimethyl-1H-pyrazol-3-yl)sulfanyl]methyl}pyridin-2-amine

The title compound was obtained by the same method as in Example 1, methods equivalent thereto or combinations of these with usual methods using butyl[4-(4,4-difluoropiperidin-1-yl)-6-{[(5-ethyl-1,4-dimethyl-1H-pyrazol-3-yl)sulfanyl]methyl}pyridin-2-yl]carbamic acid tert-butyl ester obtained in Reference Example 2-39.

¹H-NMR (CDCl₃) δ: 0.91 (3H, t, J=7.4 Hz), 1.07 (3H, t, J=7.6 Hz), 1.39 (2H, dd J=15.1, 7.6 Hz), 1.52-1.59 (2H, m), 1.84 (3H, s), 1.90-1.99 (4H, m), 2.51 (2H, q, J=7.6 Hz), 3.09-3.11 (2H, m), 3.39 (4H, t, J=5.7 Hz), 3.72 (3H, s), 3.86 (2H s), 4.60 (1H, brs), 5.54 (1H, s), 6.11 (1H, s). mass: 438(M+1)⁺.

### Example 8

### 4-(4,4-difluoropiperidin-1-yl)-6-{[(5-ethyl-1,4-dimethyl-1H-pyrazol-3-yl)sulfanyl]methyl}-N-(2-methoxyethyl)pyridin-2-amine

The title compound was obtained by the same method as in Example 1, methods equivalent thereto or combinations of these with usual methods using [4-(4,4-difluoropiperidin-1-yl)-6-{[(5-ethyl-1,4-dimethyl-1H-pyrazol-3-yl)sulfanyl]methyl}pyridin-2-yl](2-methoxyethyl)carbamic acid tert-butyl ester obtained in Reference Example 2-40.

¹H-NMR (CDCl₃) δ: 1.11 (3H, t, J=7.6 Hz), 1.88 (3H, s), 1.92-2.02 (4H, m), 2.55 (2H, q, J=7.6 Hz), 3.37 (3H, s), 3.39-3.44 (6H, m), 3.57 (2H, t, J=5.3 Hz), 3.76 (3H, s), 3.91 (2H, s), 4.70-4.77 (1H, m), 5.63 (1H, d, J=2.0 Hz), 6.16 (1H, d, J=2.0 Hz). mass: 440.3 (M+1)⁺.

### Example 9

### 6-({[4-(fluoromethyl)-5-methyl-1,3-thiazol-2-yl]sulfanyl}methyl)-N-[(6-methylpyridin-2-yl)methyl]-4-(morpholin-4-yl)pyridin-2-amine

Trifluoroacetic acid (2.00 mL) was added to [6-({[4-(hydroxymethyl)-5-methyl-1,3-thiazol-2-yl]sulfanyl}methyl)-4-(morpholin-4-yl)pyridin-2-yl][(6-methylpyridin-2-yl)methyl]carbamic acid tert-butyl ester (39.6 mg) obtained in Reference Example 2-46 at room temperature, and the mixture was stirred for 30 minutes. After concentration under reduced pressure of the reaction solution, a saturated aqueous sodium hydrogencarbonate solution was added to the residue. The mixture was extracted with ethyl acetate, and the organic layer was washed with a saturated saline solution and then dried over anhydrous sodium sulfate. Insoluble matters were filtered out, and the filtrate was concentrated under reduced pressure. The residue was dissolved in tetrahydrofuran (2 mL), followed by adding diethylaminosulfur trifluoride (18.0 μL) at 0° C. and stirring the mixture for 2 hours. A saturated aqueous sodium hydrogencarbonate solution was added to the reaction solution. The mixture was extracted with ethyl acetate, and the organic layer was washed with a saturated saline solution and then dried over anhydrous sodium sulfate. Insoluble matters were filtered out, and the filtrate was concentrated under reduced pressure. The residue was purified by thin-layer NH silica gel chromatography (hexane/ethyl acetate=25:75) to yield the title compound (10.6 mg) as a colorless oil.

¹H-NMR (CDCl₃) δ: 2.39 (3H, d, J=3.9 Hz), 2.51 (3H, s), 3.13 (4H t, J=4.9 Hz), 3.73 (4H, t, J=4.9 Hz), 4.25 (2H, s), 4.49 (2H, d, J=5.5 Hz), 5.32 (2H, d, J=48.9 Hz), 5.43 (1H, t, J=5.5 Hz), 5.62 (1H, d, J=2.0 Hz), 6.26 (1H, d, J=2.0 Hz), 6.99 (1H, d, J=7.8 Hz), 7.12 (1H, d, J=7.8 Hz), 7.49 (1H, t, J=7.8 Hz). mass: 460 (M+1)⁺.

The compounds of Examples 10 and 11 were obtained by the same method as in Example 9, methods equivalent thereto or combinations of these with usual methods using the corresponding Boc protected compounds instead of [6-({[4-(hydroxymethyl)-5-methyl-1,3-thiazol-2-yl]sulfanyl}-methyl)-4-(morpholin-4-yl)pyridin-2-yl]-[(6-methylpyridin-2-yl)methyl]carbamic acid tert-butyl ester obtained in Reference Example 2-46.

### Example 10

### 6-({[5-ethyl-4-(fluoromethyl)-1,3-thiazol-2-yl]sulfanyl}methyl)-N-[(6-methylpyridin-2-yl)methyl]-4-(morpholin-4-yl)pyridin-2-amine

The title compound was obtained by the same method as in Example 9, methods equivalent thereto or combinations of these with usual methods using [6-({[5-ethyl-4-(hydroxymethyl)-1,3-thiazol-2-yl]sulfanyl}methyl)-4-(morpholin-4-yl)pyridin-2-yl][(6-methylpyridin-2-yl)methyl]carbamic acid tert-butyl ester obtained in Reference Example 2-47.

¹H-NMR (CDCl₃) δ: 1.22 (3H, t, J=7.6 Hz), 2.51 (3H, s), 2.80 (2H, dq, J=3.5, 7.6 Hz), 3.12 (4H, t, J=4.9 Hz), 3.72 (4H, t, J=4.9 Hz), 4.26 (2H, s), 4.49 (2H, d, J=5.9 Hz), 5.32 (2H, d, J=48.9 Hz), 5.54 (1H, brs), 5.61 (1H, d, J=2.0 Hz), 6.25 (1H, d, J=2.0 Hz), 6.99 (1H, d, J=7.4 Hz), 7.12 (1H, d, J=7.8 Hz), 7.49 (1H, dd, J=7.8, 7.4 Hz). mass: 474 (M+1)⁺.

### Example 11

### 6-({[5-cyclopropyl-4-(fluoromethyl)-1,3-thiazol-2-yl]sulfanyl}methyl)-N-[(6-methylpyridin-2-yl)methyl]-4-(morpholin-4-yl)pyridin-2-amine

The title compound was obtained by the same method as in Example 9, methods equivalent thereto or combinations of these with usual methods using [6-({[5-cyclopropyl-4-(hydroxymethyl)-1,3-thiazol-2-yl]sulfanyl}methyl)-4-(morpholin-4-yl)pyridin-2-yl][(6-methylpyridin-2-yl)methyl]carbamic acid tert-butyl ester obtained in Reference Example 2-48.

¹H-NMR (CDCl₃)6:0.63-0.65 (2H, m), 1.02-1.07 (2H, m), 1.97-2.01 (1H, m), 2.51 (3H, s), 3.12 (4H, t, J=4.9 Hz), 3.72 (4H, t, J=4.9 Hz), 4.23 (2H, s), 4.49 (2H, d, J=5.5 Hz), 5.40 (2H, d, J=48.9 Hz), 5.53 (1H, brs), 5.61 (1H, d, J=2.0 Hz), 6.23 (1H, d, J=2.0 Hz), 6.99 (1H, d, J=7.8 Hz), 7.12 (1H, d, J=7.8 Hz), 7.49 (1H, t, J=7.8 Hz). mass: 486 (M+1)⁺.

Since heteroarylthiomethylpyridine derivatives according to the present invention, represented by the formula (I), or the pharmaceutically acceptable salts thereof, have a potent antagonistic action to NPY, they are useful for treatment and/or prevention of various diseases associated with NPY, for example, cardiovascular diseases such as hypertension, arteriosclerosis, nephropathy, cardiac diseases and angiospasm; diseases of central nervous system such as bulimia, depression, epilepsy, anxiety, alcoholism and dementia; metabolic diseases such as obesity, diabetes mellitus and hormone abnormality, or glaucoma. In addition, the compounds according to the present invention have a low P-glycoprotein substrate specificity and thus are excellent as medicaments. The compounds according to the present invention may be also used as PET ligands by introducing a radioactive isotope.

1. A compound represented by a formula (I) 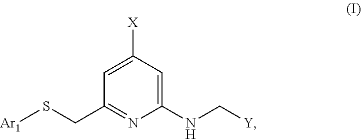
or a pharmaceutically acceptable salt thereof, wherein:
X represents a group selected from a group consisting of: 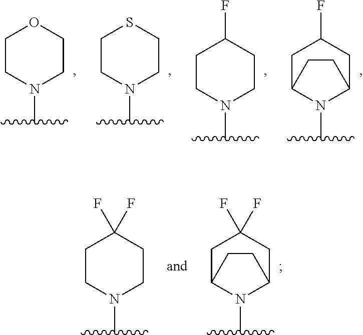
Y represents a group selected from a group consisting of: 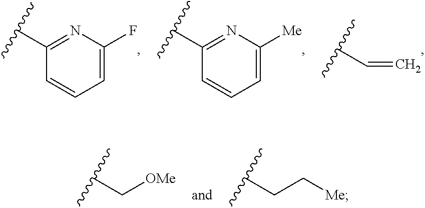
Ar₁ represents a group selected from a group consisting of:

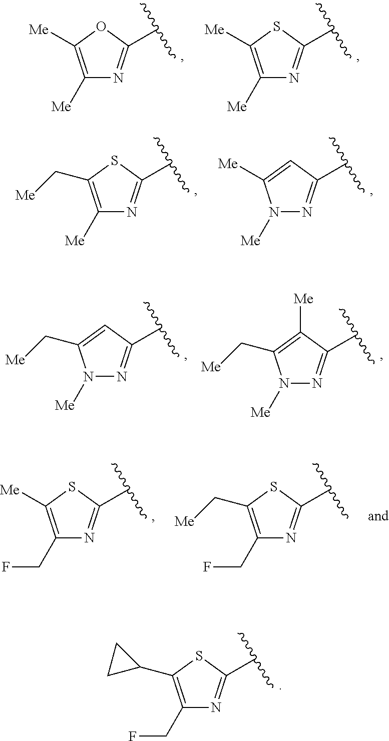

2. The compound according to claim 1,
wherein X is a group selected from 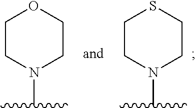
or the pharmaceutically acceptable salt thereof.

3. The compound according to claim 1, wherein Y is a group selected from 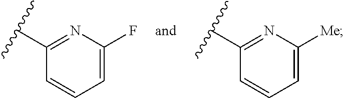
or the pharmaceutically acceptable salt thereof.

4. The compound according to claim 1, wherein Ar₁ is a group selected from 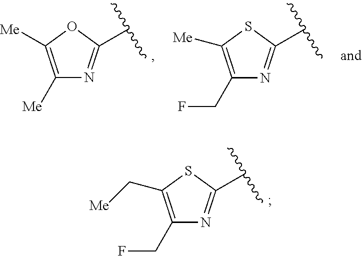
or the pharmaceutically acceptable salt thereof.

5. The compound according to claim 1, wherein the compound represented by the formula (I) is selected from the group consisting of:
6-{[(4,5-dimethyl-1,3-oxazol-2-yl)sulfanyl]methyl}-N-[(6-fluoropyridin-2-yl)methyl]-4-(thiomorpholin-4-yl)pyridin-2-amine,
6-{[(4,5-dimethyl-1,3-thiazol-2-yl)sulfanyl]methyl}-N-[(6-methylpyridin-2-yl)methyl]-4-(thiomorpholin-4-yl)pyridin-2-amine,
6-{[(5-ethyl-4-methyl-1,3-thiazol-2-yl)sulfanyl]methyl}-4-(4-fluoropiperidin-1-yl)-N-[(6-methylpyridin-2-yl)methyl]pyridin-2-amine,
6-{[(5-ethyl-4-methyl-1,3-thiazol-2-yl)sulfanyl]methyl}-4-[(3-exo)-3-fluoro-8-aza-bicyclo[3,2,1]octo-8-yl]-N-(prop-2-en-1-yl)pyridin-2-amine,
4-(3,3-difluoro-8-azabicyclo[3,2,1]octo-8-yl)-6-{[(1,5-dimethyl-1H-pyrazol-3-yl)-sulfanyl]methyl}-N-[(6-methylpyridin-2-yl)methyl]pyridin-2-amine,
4-(4,4-difluoropiperidin-1-yl)-6-{[(5-ethyl-1-methyl-1H-pyrazol-3-yl)sulfanyl]methyl}-N-(prop-2-en-1-yl)pyridin-2-amine,
N-butyl-4-(4,4-difluoropiperidin-1-yl)-6-{[(5-ethyl-1,4-dimethyl-1H-pyrazol-3-yl)-sulfanyl]methyl}pyridin-2-amine,
4-(4,4-difluoropiperidin-1-yl)-6-{[(5-ethyl-1,4-dimethyl-1H-pyrazol-3-yl)sulfanyl]methyl}-N-(2-methoxyethyl)pyridin-2-amine,
6-({[4-(fluoromethyl)-5-methyl-1,3-thiazol-2-yl]sulfanyl}methyl)-N-[(6-methylpyridin-2-yl)methyl]-4-(morpholin-4-yl)pyridin-2-amine,
6-({[5-ethyl-4-(fluoromethyl)-1,3-thiazol-2-yl]sulfanyl}methyl)-N-[(6-methylpyridin-2-yl)methyl]-4-(morpholin-4-yl)pyridin-2-amine, and
6-({[5-cyclopropyl-4-(fluoromethyl)-1,3-thiazol-2-yl]sulfanyl}methyl)-N-[(6-methyl-pyridin-2-yl)methyl]-4-(morpholin-4-yl)pyridin-2-amine.

6. The compound according to claim 1,
wherein the compound represented by the formula (I) is 6-{[(4,5-dimethyl-1,3-oxazol-2-yl)sulfanyl]methyl}-N-[(6-fluoropyridin-2-yl)methyl]-4-(thiomorpholin-4-yl)pyridin-2-amine.

7. The compound according to claim 1,
wherein the compound represented by the formula (I) is 6-({[4-(fluoromethyl)-5-methyl-1,3-thiazol-2-yl]sulfanyl}methyl)-N-[(6-methylpyridin-2-yl)methyl]-4-(morpholin-4-yl)pyridin-2-amine.

8. The compound according to claim 1,
wherein the compound represented by the formula (I) is 6-0[5-ethyl-4-(fluoromethyl)-1,3-thiazol-2-yl]sulfanyl}methyl)-N-[(6-methylpyridin-2-yl)methyl]-4-(morpholin-4-yl)pyridin-2-amine.

9. The compound according to claim 1,
wherein the human P-glycoprotein substrate specificity is low, or the pharmaceutically acceptable salt thereof.

10-11. (canceled)

12. A pharmaceutical composition comprising the compound according to claim 1, or the pharmaceutically acceptable salt thereof, and a pharmaceutically acceptable carrier.
